SEQUENCE LISTING (32 sequences)

SEQ ID NO: 1 - DNA, 20 bp, Synthetic construct
  source 1..20: mol_type = other DNA
ttgggtggatgctcacactg

SEQ ID NO: 2 - DNA, 21 bp, Synthetic construct
  source 1..21: mol_type = other DNA
gtacacgatgtctttggcaga

SEQ ID NO: 3 - DNA, 21 bp, Synthetic construct
  source 1..21: mol_type = other DNA
atgggtggacacagaatggtt

SEQ ID NO: 4 - DNA, 18 bp, Synthetic construct
  source 1..18: mol_type = other DNA
caggagcgttagtgacag

SEQ ID NO: 5 - DNA, 21 bp, Synthetic construct
  source 1..21: mol_type = other DNA
gctcttactgactggcatgag

SEQ ID NO: 6 - DNA, 20 bp, Synthetic construct
  source 1..20: mol_type = other DNA
cgcagctctaggagcatgtg

SEQ ID NO: 7 - DNA, 19 bp, Synthetic construct
  source 1..19: mol_type = other DNA
gccagaggggtttctgtcg

SEQ ID NO: 8 - DNA, 20 bp, Synthetic construct
  source 1..20: mol_type = other DNA
gttcgtgccgctaaaagtca

SEQ ID NO: 9 - DNA, 22 bp, Synthetic construct
  source 1..22: mol_type = other DNA
aacaacagctcggatttggtat

SEQ ID NO: 10 - DNA, 21 bp, Synthetic construct
  source 1..21: mol_type = other DNA
atgaccaggacattcagcagt

SEQ ID NO: 11 - DNA, 23 bp, Synthetic construct
  source 1..23: mol_type = other DNA
ggcttccttatgttcaaacaggg

SEQ ID NO: 12 - DNA, 22 bp, Synthetic construct
  source 1..22: mol_type = other DNA
gccgttactcgggtaaattaca

SEQ ID NO: 13 - DNA, 21 bp, Synthetic construct
  source 1..21: mol_type = other DNA
tggacagactaggaattggca

SEQ ID NO: 14 - DNA, 22 bp, Synthetic construct
  source 1..22: mol_type = other DNA
ccagtccgtcaacatcaaaact

SEQ ID NO: 15 - DNA, 21 bp, Synthetic construct
  source 1..21: mol_type = other DNA
gcgggagagtggaagtgaaag

SEQ ID NO: 16 - DNA, 22 bp, Synthetic construct
  source 1..22: mol_type = other DNA
gttacaaatcacagagaccgct

SEQ ID NO: 17 - DNA, 23 bp, Synthetic construct
  source 1..23: mol_type = other DNA
gactttaagggttacctgggttg

SEQ ID NO: 18 - DNA, 21 bp, Synthetic construct
  source 1..21: mol_type = other DNA
tcacatgcgccttgatgtctg

SEQ ID NO: 19 - DNA, 20 bp, Synthetic construct
  source 1..20: mol_type = other DNA
tgctctacaagggatcgggt

SEQ ID NO: 20 - DNA, 21 bp, Synthetic construct
  source 1..21: mol_type = other DNA
acacgccaaacaagaacatga

SEQ ID NO: 21 - DNA, 21 bp, Synthetic construct
  source 1..21: mol_type = other DNA
agcagcaattaagaactgcca

SEQ ID NO: 22 - DNA, 21 bp, Synthetic construct
  source 1..21: mol_type = other DNA
taccaaagccgagctgttgtt

SEQ ID NO: 23 - DNA, 20 bp, Synthetic construct
  source 1..20: mol_type = other DNA
gacgctgtctttgcataggc

SEQ ID NO: 24 - DNA, 23 bp, Synthetic construct
  source 1..23: mol_type = other DNA
ggatttaggcatcgttgtccttt

SEQ ID NO: 25 - DNA, 21 bp, Synthetic construct
  source 1..21: mol_type = other DNA
cctgaaccttccaaagatggc

SEQ ID NO: 26 - DNA, 21 bp, Synthetic construct
  source 1..21: mol_type = other DNA
ttcaccaggcaagtctcctca

SEQ ID NO: 27 - DNA, 19 bp, Synthetic construct
  source 1..19: mol_type = other DNA
gaggccaagccctggtatg

SEQ ID NO: 28 - DNA, 19 bp, Synthetic construct
  source 1..19: mol_type = other DNA
cgggccgattgatctcagc

SEQ ID NO: 29 - DNA, 19 bp, Synthetic construct
  source 1..19: mol_type = other DNA
gtcagcgagaacaaggtgc

SEQ ID NO: 30 - DNA, 21 bp, Synthetic construct
  source 1..21: mol_type = other DNA
gatccgctcagccgtattcat

SEQ ID NO: 31 - DNA, 21 bp, Synthetic construct
  source 1..21: mol_type = other DNA
tctggcggcttgaagaatttg

SEQ ID NO: 32 - DNA, 23 bp, Synthetic construct
  source 1..23: mol_type = other DNA
ggtgatcggagtatagttggagc

(19) United States
(12) Patent Application Publication
(10) Pub. No.: US 2025/0188457 A1
(43) Pub. Date: Jun. 12, 2025

(54) COMPOSITIONS AND METHODS FOR THE TREATMENT OF GLIOBLASTOMA BY INHIBITION OF THE TFPI2-CD51-STAT6 SIGNALING AXIS

(71) Applicant: Northwestern University, Evanston, IL (US)

(72) Inventors: Peiwen CHEN, Evanston, IL (US); Lizhi PANG, Evanston, IL (US)

(21) Appl. No.: 18/885,350

(22) Filed: Sep. 13, 2024

Related U.S. Application Data
(60) Provisional application No. 63/582,715, filed on Sep. 14, 2023.

(51) Int. Cl. C12N 15/113; A61K 31/4375; A61K 31/505; A61K 39/00; A61P 35/00; C07K 16/28

(52) U.S. Cl. CPC C12N 15/113; A61K 31/4375; A61K 31/505; A61P 35/00; C07K 16/2818; A61K 2039/505; C12N 2310/11; C12N 2310/3233; C12N 2310/531

(57) ABSTRACT

Provided herein are inhibitors of the TFPJ2-CD51-STAT6 signaling axis and methods for the treatment of cancer (e.g., glioblastoma) therewith. In particular, inhibitors of expression or activity of components of the TFPJ2-CD51-STAT6 signaling axis are provided, with or without an immune checkpoint inhibitor, for the treatment of glioblastoma.

### CROSS-REFERENCE TO RELATED APPLICATION

The present application claims priority to U.S. Provisional Patent Application Ser. No. 63/582,715 filed Sep. 14, 2023, which is hereby incorporated by reference in its entirety.

### STATEMENT REGARDING FEDERAL FUNDING

This invention was made with Government support under grant numbers NS124594 and CA240896 awarded by the National Institutes of Health, and grant number W81XWH-21-1-0380 awarded by the Department of Defense. The government has certain rights in the invention.

### SEQUENCE LISTING

The text of the computer readable sequence listing filed herewith, titled “NWEST-42252_202_SequenceListing.xml”, created Sep. 13, 2024, having a file size of 29,261 bytes, is hereby incorporated by reference in its entirety.

### FIELD

Provided herein are inhibitors of the TFPI2-CD51-STAT6 signaling axis and methods for the treatment of cancer (e.g., glioblastoma) therewith. In particular, inhibitors of expression or activity of components of the TFPI2-CD51-STAT6 signaling axis are provided, with or without an immune checkpoint inhibitor, for the treatment of glioblastoma.

### BACKGROUND

Glioblastoma (GBM) is a devastating primary brain tumor. The median survival of GBM patients is approximately 14-16 months following initial diagnosis despite aggressive standard-of-care (SOC) treatment, which includes maximal surgical resection followed by chemo/radiotherapy (Ref. 1; incorporated by reference in its entirety). Poor prognosis and treatment recurrence of GBM are in part due to the presence of glioblastoma stem cells (GSCs), a subpopulation of cancer cells that are featured by stem cell-like capabilities (Ref. 2; incorporated by reference in its entirety). Mice implanted with GSCs produce tumors that are less responsive to chemotherapy than tumors established from more differentiated GBM cells: a finding that supports a crucial role of GSCs in treatment resistance (Ref. 3; incorporated by reference in its entirety). GSCs only account for ˜10% of total cancer cells in tumor tissues. However, the GSC subpopulation is known for self-renewal that is important for tumor growth and recurrence. Given the significant inverse correlation between GSC stemness and survival (Ref. 4; incorporated by reference in its entirety), targeting GSCs holds a great potential for GBM therapy (Refs. 2, 5-6; incorporated by reference in its entirety).

Despite advances in the understanding of GSC biologic properties, significant knowledge gaps remain, including those involving relationships between GSCs and additional cell populations of the tumor microenvironment (TME) (Refs. 2, 7; incorporated by reference in its entirety). Within the GBM TME, tumor-associated macrophages and microglia (TAMs) are abundant and account for up to 50% of total cells in the tumor mass (Ref. 8; incorporated by reference in its entirety). TAMs are usually polarized towards an immunosuppressive phenotype that can inhibit the infiltration and anti-tumor activation of cytotoxic T cells and induce immunotherapy resistance. Increasing evidence supports a symbiotic interaction between GSCs and TAMs (Refs. 2, 7, 9-10; incorporated by reference in their entireties). For instance, recent studies have demonstrated that GSC expression of CLOCK and its heterodimeric partner BMAL1 transcriptionally upregulates olfactomedin-like 3 (OLFML3) and legumain (LGMN) that, in turn, promote microglia infiltration and immunosuppressive polarization (Refs. 11-12; incorporated by reference in their entireties).

### SUMMARY

Provided herein are inhibitors of the TFPI2-CD51-STAT6 signaling axis and methods for the treatment of cancer (e.g., glioblastoma) therewith. In particular, inhibitors of expression or activity of components of the TFPI2-CD51-STAT6 signaling axis are provided, with or without an immune checkpoint inhibitor, for the treatment of glioblastoma.

In some embodiments, provided herein are methods of treating cancer in a subject, wherein the subject has an inhibited TFPI2-CD51-STAT6 signaling axis, the method comprising administering to the subject an immune checkpoint inhibitor (ICI). In some embodiments, the subject has been previously administered a TFPI2-CD51-STAT6 signaling axis inhibitor (e.g., TFPI2 inhibitor).

In some embodiments, provided herein are methods of treating cancer in a subject comprising administering a TFPI2 inhibitor to the subject.

In some embodiments, provided herein are methods of treating cancer in a subject comprising co-administering to the subject: (a) a TFPI2-CD51-STAT6 signaling axis inhibitor, and (b) an immune checkpoint inhibitor (ICI).

In some embodiments, the subject suffers from glioblastoma.

In some embodiments, an ICI targets an immune checkpoint molecule/protein selected from the group consisting of CTLA4, PD-1, PD-L1, PD-L2, A2AR, B7-H3, B7-H4, BTLA, KIR, LAG3, TIM-3, VISTA, or a ligand/receptor thereof. In some embodiments, an ICI is selected from nivolumab, pembrolizumab, atezolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

In some embodiments, a TFPI2-CD51-STAT6 signaling axis inhibitor is a STAT6 inhibitor. In some embodiments, the STAT6 inhibitor is an inhibitor of STAT6 expression. In some embodiments, the STAT6 inhibitor is an inhibitor of STAT6 activity.

In some embodiments, a TFPI2-CD51-STAT6 signaling axis inhibitor is a CD51 inhibitor. In some embodiments, the CD51 inhibitor is an inhibitor of CD51 expression. In some embodiments, the CD51 inhibitor is an inhibitor of CD51 activity.

In some embodiments, a TFPI2-CD51-STAT6 signaling axis inhibitor is a TFPI2 inhibitor. In some embodiments, the TFPI2 inhibitor is an inhibitor of TFPI2 expression. In some embodiments, the TFPI2 inhibitor is an inhibitor of TFPI2 activity.

In some embodiments, an inhibitor of TFPI2, CD51, or STAT6 expression is an shRNA, a miRNA, a morpholino, a ribozyme, an antisense nucleic acid molecule, or a CRISPR-based construct.

In some embodiments, an inhibitor of TFPI2, CD51, or STAT6 activity is a small molecule, peptide, antibody, or antibody fragment.

In some embodiments, administration or co-administration is by one or more of oral, intravenous, or other acceptable routes.

### BRIEF DESCRIPTION OF THE DRAWINGS

FIG. 1A-O. TFPI2 overexpression promotes GSC self-renewal. a, Correlation analysis between the GSC signature and immune/stromal scores in The Cancer Genome Atlas (TCGA) GBM dataset (n=300). R and P values are shown. Pearson test. b, Identification of the two genes (TFPI2 and LOXL2) that encode secreted proteins and are correlated negatively with patient survival and positively with the sternness signature in TCGA GBM patient tumors, and highly expressed in CD45⁻ cells of GBM tumors. c, Amplification of TFPI2 and LOXL2 in GBM and low-grade glioma (LGG) patient tumors. d, TFPI2 copy number is significantly correlated with TFPI2 mRNA expression level in TCGA GBM patient tumors under the Agilent-4502A platform. e, Immunoblot for TFPI2 in cell lysates of different GBM patient-derived GSCs (e.g., GSC262, GSC17, GSC2, GSC272, GSC20, and GSC23; upper panel) and mouse-derived GSCs and GBM cells (e.g., QPP7, 005 GSC, CT2A, SB28, GL261; bottom panel). Based on the expression levels, GSC272, GSC2, QPP7 GSC, CT2A, and 005 GSC are considered as TFPI2-high cells. f,g, Immunoblots for TFPI2, CD133, and SOX2 in cell lysates of GSC262 (f) and GSC23 (g) cells with or without TFPI2 overexpression. h,i, Immunoblots for CD133 and SOX2 in cell lysates of GSC262 (h) and GSC23 (i) cells treated with or without TFPI2 recombinant protein at indicated concentrations for 24 hrs. j, Representative of tumorspheres of GSC262 and GSC23 cells expressing control and TFPI2 overexpression (OE) plasmid. Scale bar, 400 mm. k,l, Quantification of tumorspheres of GSC262 (k) and GSC23 (l) cells expressing control and TFPI2 overexpression (OE) plasmid. n=16-33 biological replicates. m, Representative of tumorspheres of GSC262 and GSC23 cells treated with TFPI2 recombinant protein at indicated concentrations. Scale bar, 400 mm. n,o, Quantification of tumorspheres of GSC262 (n) and GSC23 (o) cells treated with TFPI2 recombinant protein at indicated concentrations. n=12-21 biological replicates. Data from multiple replicates are presented as mean. Error bars indicate mean±SD or SEM. One-way ANOVA test.

FIG. 2A-T. Genetic depletion of TFPI2 impairs GSC self-renewal and proliferation, and extends survival of GSC-bearing mice. a,b, Immunoblots for TFPI2 (a), CD133 and SOX2 (b) in cell lysates from human GSC272, and mouse QPP7 GSCs and CT2A cells expressing shRNA control (shC) and TFPI2 shRNAs (shTFPI2). c,d, Representative images (c) and quantification (d) of tumorspheres in GSC272 cells expressing shC and shTFPI2. n=6 biological replicates. Scale bar, 200 mm. e,f, Quantification of tumorspheres in QPP7 GSCs (e) and CT2A cells (f) expressing shC and shTfpi2. n=6-7 biological replicates. g,h, Representative images (g) and quantification (h) of proliferation in GSC272 cells expressing shC and shTFPI2. The percentage of proliferating cells for each group is indicated. n=3 biological replicates. i,j, Quantification of proliferation in QPP7 GSCs (i) and CT2A cells (j) expressing shC and shTfpi2. n=3 biological replicates. k, Quantification of tumorspheres in QPP7 GSCs expressing shTfpi2 with or without reexpression of shRNA-resistant TFPI2 cDNA. n=6 biological replicates. l, Quantification of tumorspheres in TFPI2 wild-type (WT) and CRISPR knockout (KO) GSC272 cells. n=12 biological replicates. m,n, Survival curves of C57BL/6 mice implanted with 2×10⁵ (m) or 2×10⁴ (n) QPP7 GSCs expressing shC and shTfpi2. n=11 biological replicates. o, Survival curves of C57BL/6 mice implanted with 2×10⁴ CT2A cells expressing shC and two independent Tfpi2 shRNAs. n=7-9 biological replicates. p, Survival curves of nude mice implanted with 2×10⁵ GSC272 cells expressing shC and two independent TFPI2 shRNAs. n=9-10 biological replicates. q, Representative of immunofluorescence staining of SOX2, Ki67, and cleaved caspase 3 (CC3) in tumors from C57BL/6 mice intracranially implanted with QPP7 GSCs expressing shC and shTfpi2. Scale bar, 50 mm. r-t, Quantification of the relative expression levels of SOX2 (r), Ki67 (s), and CC3 (t) in tumors from C57BL/6 mice intracranially implanted with QPP7 GSCs expressing shC and shTfpi2. n=3 biological replicates. Data from multiple replicates are presented as mean. Error bars indicate mean±SEM. One-way ANOVA test. In (m) to (p), log-rank test.

FIG. 3A-O. TFPI2 promotes GSC self-renewal and tumor growth through activation of the JNK-STAT3 signaling pathway. a, Transcriptomic profiling in GSC272 cells following TFPI2 depletion and GSEA analysis shows the eight oncogenic pathways affected by TFPI2 depletion. b, Immunoblots for P-JNK and JNK in GSC23 and GSC262 cells expressing control and TFPI2 overexpression (OE) plasmid. c, Immunoblots for P-JNK and JNK in GSC272, QPP7 GSCs, and CT2A cells expressing shRNA control (shC) and TFPI2 shRNAs (shTFPI2). d, Representative images of tumorspheres in GSC23 and GSC262 expressing control and TFPI2 OE plasmid treated with or without JNK inhibitor JNK-IN-8 (10 nM). Scale bar, 400 mm. e,f, Quantification of tumorspheres in GSC23 (e) and GSC262 (f) expressing control and TFPI2 OE plasmid treated with or without JNK-IN-8 (10 nM). n=6 biological replicates. g, Identification of two oncogenic pathways (as indicated) in distinct datasets with three comparisons (GSC272 RNA-seq data: shC versus shTFPI2 and two TCGA GBM datasets: TFPI2-high versus TFPI2-low). h, Immunoblots for P-STAT3 and STAT3 in GSC23 and GSC262 expressing control and TFPI2 OE plasmid. i, Immunoblots for P-STAT3 and STAT3 in GSC272, QPP7 GSCs, and CT2A cells expressing shC and shTFPI2. j, Representative images of tumorspheres in GSC23 and GSC262 expressing control and TFPI2 OE plasmid treated with or without STAT3 inhibitor WP1066 (20 nM). Scale bar, 400 mm. k,l, Quantification of tumorspheres in GSC23 (k) and GSC262 (l) expressing control and TFPI2 OE plasmid treated with or without WP1066 (20 nM). n=6 biological replicates. m, Immunoblots for P-STAT3 and STAT3 in GSC23 and GSC262 expressing control and TFPI2 OE plasmid treated with or without JNK inhibitor JNK-IN-8 (10 nM). n, Immunoblots for P-JNK and JNK in GSC23 and GSC262 expressing control and TFPI2 OE plasmid treated with or without STAT3 inhibitor WP1066 (20 nM). o, Survival curves of nude mice implanted with 2×10⁴ GSC23 expressing control and TFPI2 overexpression (OE) plasmid. Mice were treated with JNK inhibitor JNK-IN-8 (30 mg/kg body weight, i.p., every day) or STAT3 inhibitor WP1066 (30 mg/kg body weight, i.p., every day) on day 7. n=6 biological replicates. Data from multiple replicates are presented as mean. Error bars indicate mean±SEM. One-way ANOVA test. In (o), log-rank test.

FIG. 4A-S. TFPI2 activates STAT6 to promote microglia infiltration. a,b, Representative images (a) and quantification (b) of relative migration of mouse SIM-A9 microglia, human HMC3 microglia, and primary human microglia (PrhMG) following stimulation with TFPI2 recombinant protein (20 ng/ml). Scale bars, 200 mm for HMC3 and SIM-A9, and 400 mm for PrhMG. n=3 biological replicates. c, Quantification of transwell migration of PrhMG following stimulation with conditioned media (CM) from GSC262 or GSC23 cells expressing control and three independent TFPI2 overexpression (OE) plasmid clones. n=3 biological replicates. d, Quantification of transwell migration of PrhMG, HMC3 and SIM-A9 following stimulation with CM from GSC272 cells (for PrhMG and HMC3) or QPP7 cells (for SIM-A9) expressing control shRNA (shC) and TFPI2 shRNAs (shTFPI2). n=3 biological replicates. e,f, Representative images (e) and quantification (f) of flow cytometry analysis for the percentage of CD45^{low}CD11b⁺CX3CR1⁺ microglia in size matched tumors from C57BL/6 mice intracranially implanted with CT2A cells expressing shC and shTfpi2. n=3-4 biological replicates. g,h, Immunofluorescence (g) and quantification (h) of CX3CR1 in size matched tumors from C57BL/6 mice intracranially implanted with QPP7 GSCs expressing shC and shTfpi2. Scale bar, 25 μm. n=3 biological replicates. i,j, Immunofluorescence (i) and quantification (j) of CX3CR1 in size matched tumors from nude mice intracranially implanted with GSC272 expressing shC and shTFPI2. Scale bar, 25 μm. n=6 biological replicates. k, Representative of human phospho-kinase antibody array experiment in HMC3 cells treated with or without 20 ng/ml TFPI2 recombinant protein. Affected kinases are indicated. l,m, Immunoblots for P-STAT6 and STAT6 in cell lysates of HMC3 microglia (l), as well as SIM-A9 microglia and PrhMG (m) incubated with TFPI2 recombinant protein at indicated concentrations and time points. n,o, Representative images (n) and quantification (o) of flow cytometry analysis for P-STAT6 expression in PrhMG treated with or without TFPI2 recombinant protein (20 ng/ml). n=3 biological replicates. p, Quantification of transwell migration of mouse primary microglia isolated from wild-type (WT) and STAT6 knockout (KO) mice following stimulation with TFPI2 recombinant protein (20 ng/ml). n=3 biological replicates. q, Quantification of transwell migration of PrhMG, HMC3, and SIM-A9 microglia following stimulation with TFPI2 recombinant protein (20 ng/ml) in the presence or absence of STAT6 inhibitor AS1517499 at indicated concentrations. n=3 biological replicates. r,s, Quantification of incucyte live-cell (r) and scratch wound healing (s) migration of HMC3 microglia following stimulation with TFPI2 recombinant protein (20 ng/ml) in the presence or absence of STAT6 inhibitor (STAT6i) AS1517499 (25 nM). n=3 biological replicates. Data from multiple replicates are presented as mean. Error bars indicate mean±SEM. Student's t-test and one-way ANOVA test.

FIG. 5A-S. TFPI2-STAT6 signaling axis triggers microglia immunosuppressive polarization. a,b, Representative (a) and quantification (b) of flow cytometry analysis for the percentage of CD206⁺ PrhMG treated with conditioned media (CM) from GSC262 cells expressing control and TFPI2 overexpression (OE) plasmid. n=3 biological replicates. c, Quantification of flow cytometry analysis for the percentage of CD206⁺ PrhMG treated with CM from GSC23 cells expressing control and TFPI2 OE plasmid. n=3 biological replicates. d, Quantification of flow cytometry analysis for the percentage of CD206⁺ HMC3 microglia treated with CM from GSC272 cells expressing shRNA control (shC) and TFPI2 shRNAs (shTFPI2). n=3 biological replicates. e, Quantification of flow cytometry analysis for the percentage of CD206⁺ SIM-A9 microglia treated with CM from QPP7 GSCs expressing shC and shTfpi2 with or without reexpression of shRNA-resistant TFPI2 cDNA (OE). n=3 biological replicates. f,g, Representative images (f) and quantification (g) of flow cytometry analysis for the percentage of CD45^{low}CD11b⁺CX3CR1⁺CD206⁺ microglia in size matched tumors from C57BL/6 mice intracranially implanted with CT2A cells expressing shC and shTfpi2. n=3-4 biological replicates. h-j, Immunofluorescence (h) and quantification of CX3CR1⁺ CD206⁺ (i) or CX3CR1⁺CD163⁺ (j) cells in size matched tumors from C57BL/6 mice intracranially implanted with QPP7 GSCs expressing shC and shTfpi2. Scale bar, 25 □m. n=6 biological replicates. k, Quantification of flow cytometry analysis for the percentage of CD206⁺ cells in mouse primary microglia isolated from wild-type (WT) and STAT6 knockout (KO) mice following stimulation with TFPI2 recombinant protein (20 ng/ml). n=3 biological replicates. l,m, Representative images (l) and quantification (m) of flow cytometry analysis for arginase 1 (ARG1) expression in mouse primary microglia isolated from WT and STAT6 KO mice following stimulation with TFPI2 recombinant protein (20 ng/ml). n=3 biological replicates. n,o, Quantification of flow cytometry analysis for the percentage of CD206⁺ cells (n) and ARG1 expression (o) in SIM-A9 microglia treated with TFPI2 recombinant protein (20 ng/ml) in the presence or absence of STAT6i AS1517499 (25 nM). n=3 biological replicates. p,q, Quantification of flow cytometry analysis for the percentage of CD206⁺ cells (p) and ARG1 expression (q) in HMC3 microglia treated with TFPI2 recombinant protein (20 ng/ml) in the presence or absence of STAT6i AS1517499 (25 nM). n=3 biological replicates. r, Relative mRNA expression of Arg1, Cd163, Il0, Mif, 1123, and Cxcl11 in SIM-A9 cells treated with TFPI2 recombinant protein (20 ng/ml) in the presence or absence of STAT6i AS1517499 (25 nM). n=3 biological replicates. s, Relative mRNA expression of ARG1, CD163, IL10, MRC1, MIF, IL23, and CXCL11 in HMC3 cells treated with TFPI2 recombinant protein (20 ng/ml) in the presence or absence of STAT6i AS1517499 (25 nM). n=3-4 biological replicates. Data from multiple replicates are presented as mean. Error bars indicate mean±SEM. One-way ANOVA test.

FIG. 6A-Q. CD51 is a functional receptor that mediates TFPI2-induced microglia migration and immunosuppressive polarization. a, Working flow for identifying TFPI2 receptor on microglia. TFPI2-associated membrane proteins from PrhMG were immunoprecipitated. By overlapping TFPI2 antibody-enriched proteins (FC>2) with human membrane protein dataset, 15 receptor candidates were identified. CD51 is the only candidate that highly expressed in microglia with respective to other cell types in GBM using Brain Tumor Immune Micro Environment (TIME) dataset. b,c, Immunoprecipitation with CD51 antibody (CD51 Ab) and immunoblotting for the interaction between TFPI2 and CD51 in SIM-A9 (b) and HMC3 (c) microglia. d, Calcium mobilization triggered by TFPI2 recombinant protein (20 ng/ml) in SIM-A9 microglia expressing shRNA control (shC) or Itgav shRNAs (shItgav) e, Calcium mobilization triggered by TFPI2 recombinant protein (20 ng/ml) in SIM-A9 microglia treated with or without CD51 inhibitor MK-0429 at indicated concentrations. f, Immunoblots for P-PLCγ1, PLCγ1, P-PKCz, and PKCz in cell lysates of HMC3 microglia incubated with TFPI2 recombinant protein (20 ng/ml) in the presence or absence of MK-0429 (50 nM). g, Immunoblots for P-STAT6 and STAT6 in cell lysates from SIM-A9 microglia expressing shC and shItgav treated with or without TFPI2 recombinant protein (20 ng/ml). h, Immunoblots for P-STAT6 and STAT6 in cell lysates from HMC3 microglia treated with or without TFPI2 recombinant protein (20 ng/ml) and MK-0429 (50 nM). i,j, Representative images (i) and quantification (j) of relative migration of mouse SIM-A9 microglia expressing shC and shItgav following stimulation with TFPI2 recombinant protein (20 ng/ml). Scale bars, 400 mm. n=3 biological replicates. k, Quantification of relative migration of PrhMG, human HMC3, and mouse SIM-A9 microglia treated with or without TFPI2 recombinant (20 ng/ml) in the presence or absence of MK-0429 at indicated concentrations. l,m, Representative (l) and quantification (m) of flow cytometry analysis for the percentage of CD206⁺ SIM-A9 microglia expressing shC and shItgav following stimulation with or without TFPI2 recombinant protein (20 ng/ml). n=3 biological replicates. n,o, Representative (n) and quantification (o) of flow cytometry analysis for the percentage of ARG1⁺ SIM-A9 microglia expressing shC and shItgav following stimulation with or without TFPI2 recombinant protein (20 ng/ml). n=3 biological replicates. p,q, Quantification of flow cytometry analysis for the percentage of CD206⁺ (p) and ARG1⁺ (q) PrhMG treated with TFPI2 recombinant protein (20 ng/ml) in the presence or absence of MK-0429 (50 nM). n=3 biological replicates. Data from multiple replicates are presented as mean. Error bars indicate mean±SEM. One-way ANOVA test.

FIG. 7A-U. Inhibition of the TFPI2-CD51-STAT6 signaling axis activates antitumor immune response and synergizes with anti-PD1 therapy. a,b, Representative (a) and quantification (b) of flow cytometry analysis for the percentage of CD45⁺CD3⁺ T cells in the spleens of C57BL/6 mice bearing size matched CT2A tumors expressing shRNA control (shC) and Tfpi2 shRNAs (shTfpi2). n=3-4 biological replicates. c-f, Quantification of flow cytometry analysis for the percentage of CD45⁺CD3⁺CD8⁺ T cells (c), CD45⁺CD3⁺CD4⁺ T cells (d) CD45⁺CD3⁺CD8⁺PD1^{hi} T cells (e), and CD45⁺CD3⁺CD4⁺PD1^{hi} T cells (f) in the spleens of C57BL/6 mice bearing size matched CT2A tumors expressing shC and shTfpi2. n=3-4 biological replicates. g,h, Representative (g) and quantification (h) of the percentage of fixable viability dye-labeled mouse primary CD8⁺ T cells cocultured with SIM-A9 microglia treated with TFPI2 recombinant protein (20 ng/ml) in the presence or absence of STAT6 inhibitor (STAT6i) AS1517499 (25 nM). n=3 biological replicates. i,j, Representative (i) and quantification (j) of flow cytometry analysis for the percentage of CD69⁺ cells out of total CD8⁺ T cells cocultured with SIM-A9 microglia treated with TFPI2 recombinant protein (20 ng/ml) in the presence or absence of STAT6i AS1517499 (25 nM). n=3 biological replicates. k, Quantification of flow cytometry analysis for the percentage of IFNγ⁺ cells out of total CD8⁺ T cells cocultured with SIM-A9 microglia treated with TFPI2 recombinant protein (20 ng/ml) in the presence or absence of STAT6i AS1517499 (25 nM). n=3 biological replicates. 1-n, Quantification of flow cytometry analysis for the percentage of fixable viability dye-labeled cells (l), CD69⁺ cells (m), and IFNγ⁺ cells (n) out of total JURKAT T cells cocultured with PrhMG treated with TFPI2 recombinant protein (20 ng/ml) in the presence or absence of STAT6i AS1517499 (25 nM). n=3 biological replicates. o, CT2A cancer cell cytotoxicity induced by activated mouse primary CD8⁺ T cells (the ratios between CD8⁺ T cells and CT2A cells are indicated) cocultured with SIM-A9 microglia treated with TFPI2 recombinant protein (20 ng/ml) in the presence or absence of STAT6i AS1517499 (25 nM). n=10 biological replicates. p, GSC272 cell cytotoxicity induced by activated JURKAT T cells (the ratios between JURKAT T cells and GSC272 cells are indicated) cocultured with PrhMG treated with TFPI2 recombinant protein (20 ng/ml) in the presence or absence of STAT6i AS1517499 (25 nM). n=10 biological replicates. q, Survival curves of C57BL/6 mice implanted with CT2A cells (2×10⁴ cells/mouse) expressing shC and shTfpi2. Mice were treated with IgG or anti-PD1 (10 mg/kg body weight, i.p.) on days 11, 14, and 17. n=6-8 biological replicates. r,s, Survival curves of C57BL/6 mice implanted with 005 GSCs (2×10⁵ cells/mouse; r) or CT2A cells (2×10⁴ cells/mouse; s). Mice were treated with CD51 inhibitor MK-0429 (30 mg/kg, i.p., every other day) on day 7, and then received the treatment with IgG or anti-PD1 (10 mg/kg body weight, i.p.) on days 11, 14, and 17. n=7-10 biological replicates. t,u, Survival curves of C57BL/6 mice implanted with 005 GSCs (2×10⁵ cells/mouse; t) or CT2A cells (2×10⁴ cells/mouse; u). Mice were treated with STAT6i AS1517499 (10 mg/kg, i.p., every other day) on day 7, and then received the treatment with IgG or anti-PD1 (10 mg/kg body weight, i.p.) on days 11, 14, and 17. n=6 biological replicates. Data from multiple replicates are presented as mean. Error bars indicate mean±SEM. One-way ANOVA test. In (q) to (u), log-rank test.

FIG. 8A-L. TFPI2 is a prognostic biomarker, correlates with GSC sternness and microglia abundance in GBM patients. a, Representative images show low, medium, and high expression of TFPI2, CD133, SOX2, and CX3CR1 in human GBM tumor samples based on immunohistochemistry staining. Scale bar, 100 mm. b-d, Quantification of immunohistochemistry staining showing strong positive correlations between TFPI2 and CD133 (b), TFPI2 and SOX2 (c), or TFPI2 and CX3CR1 (d) in human GBM tumor samples (n=60). R and P values are shown. Pearson test. e, Quantification of microglia signature in GBM patient tumors with GBM cell TFPI2 high and low based on single cell RNA-seq data (EGAS00001004422 and GSE131928). f-h, GSEA for stemness signature (f), microglia signature (g), and activated CD8⁺ T cell signature (h) in TFPI2-high patient tumors compared with TFPI2-low patient tumors from TCGA GBM dataset. i, ELISA for TFPI2 in the plasma from healthy controls (n=6) and GBM patients (n=55). j, Correlation of relative Ki67 expression in tumors and TFPI2 plasma concentration in GBM patients (n=55). R and P values are shown. Pearson test. k, Correlation of overall survival and TFPI2 plasma concentration in GBM patients (n=35). R and P values are shown. Pearson test. l, Overall survival of patients with high (n=23) and low (n=31) TFPI2 plasma concentration (Cut-off at median). Log-rank P value is shown. Data from multiple replicates are presented as mean. Error bars indicate mean±SEM. Student's t-test.

FIG. 9A-R. TFPI2 correlates with GSC stemness, GBM tumor grades, and survival, and promotes GSC self-renewal. a, Correlation of TFPI2 or LOXL2 gene expression and IDH1 WT GBM patient survival in TCGA-GBM RNA-Seq dataset and TCGA-GBM-LGG dataset. Log-rank test, Wilcoxon signed-rank test, and Pearson test were performed. P values are shown. b, Correlation of stemness signature and TFPI2 or LOXL2 mRNA expression and in TCGA GBM dataset. R and P values are shown. Pearson test. c, Correlation of GSC signature and TFPI2 or LOXL2 mRNA expression and in TCGA GBM dataset. R and P values are shown. Pearson test. d,e, Expression of TFPI2 (d) and LOXL2 (e) in CD45⁻ cells and immune cells, including microglia (MG), monocyte-derived macrophages (MDM), neutrophils, CD4⁺ T cells, and CD8⁺ T cells in non-tumor tissues and IDH1-WT GBM tumors. f, TFPI2 copy number is significantly correlated with TFPI2 mRNA expression in TCGA GBM patient tumors under the HG-U133A platform. g, TFPI2 expression in glioma tumors of different stages. h, TFPI2 expression in different types of brain tumors. OG, oligodendroglioma; OA, oligoastrocytoma. i, TFPI2 expression in different subtypes of GBM tumors. j,k, High-resolution UMAP dimensional reduction of all cells (j) and malignant cells (k) from brain tumor samples of a cohort of GBM patients. l,m, UMAP dimensional reduction of the basis of the expression pattern of hallmark stemness signature (l) and TFPI2 (m) in malignant cells. n, Quantification of hallmark stemness signature in TFPI2-low and TFPI2-high tumors. o, Immunoblots for CD133 and SOX2 in cell lysates from GSC20 cells treated with or without TFPI2 recombinant protein at indicated concentrations. p, Representative of flow cytometry analysis of apoptosis in GSC262 and GSC23 cells expressing control and TFPI2 overexpression (OE) plasmid. Cells were labeled with propidium iodide (PI) and Apotracker (FITC) for flow cytometry analysis. q,r, Quantification of apoptosis in GSC262 (q) and GSC23 (r) cells expressing control and TFPI2 OE plasmid. n=3 biological replicates. Data from multiple replicates are presented as mean. Error bars indicate mean±SD (f-i) or SEM (q, r). One-way ANOVA test (f-i, q and r) and Student's t-test (n).

FIG. 10A-X. Genetic depletion of TFPI2 impairs GSC self-renewal and proliferation and promotes apoptosis in vitro and in vivo. a,b, Representative of tumorspheres of QPP7 (a) and CT2A (b) cells expressing control shRNA (shC) and Tfpi2 shRNAs (shTfpi2). Scale bar, 200 mm. c,d, Representative images of proliferation in QPP7 (c) and CT2A (d) cells expressing shC and shTfpi2. The percentage of proliferating cells for each group is indicated. e,f, Representative (e) and quantification (f) of flow cytometry analysis of apoptosis in GSC272 cells expressing shC and shTFPI2. Cells were labeled with propidium iodide (PI) and Apotracker (FITC) for flow cytometry analysis. n=3 biological replicates. g,h, Representative (g) and quantification (h) of flow cytometry analysis of apoptosis in QPP7 cells expressing shC and shTfpi2. Cells were labeled with PI and Apotracker (FITC) for flow cytometry analysis. n=3 biological replicates. i,j, Representative (i) and quantification j) of flow cytometry analysis of apoptosis in CT2A cells expressing shC and shTfpi2. Cells were labeled with PI and Apotracker (FITC) for flow cytometry analysis. n=3 biological replicates. k, Immunoblots for cleaved caspase 3 (CC3) in cell lysates from GSC272, QPP7, and CT2A cells expressing shC and shTFPI2. l, Immunoblots for CD133 and SOX2 in cell lysates from QPP7 GSCs expressing shTfpi2 with or without reexpression of shRNA-resistant TFPI2 cDNA (TFPI2 OE). m, Representative images of tumorspheres in QPP7 GSCs expressing shTfpi2 with or without TFPI2 OE. Scale bar, 400 mm. n, Immunoblots for TFPI2 in cell lysates from TFPI2 wild-type (WT) and different clones of CRISPR knockout (KO) GSC272 cells. o, Immunoblots for CD133 and SOX2 in cell lysates from TFPI2 WT and CRISPR KO GSC272 cells. p, Representative images of tumorspheres in TFPI2 WT and CRISPR KO GSC272 cells. Scale bar, 400 mm. q,r, Representative (q) and quantification (r) of immunofluorescence staining of SOX2 in size-matched tumors from C57BL/6 mice intracranially implanted with CT2A cells expressing shC and shTfpi2. Scale bar, 50 mm. n=3 biological replicates. s,t, Representative (s) and quantification (t) of immunochemistry staining of CD133 in tumors from C57BL/6 mice intracranially implanted with CT2A cells expressing shC and shTfpi2. Scale bar, 200 mm. n=3 biological replicates. u,v, Representative (u) and quantification (v) of immunofluorescence staining of Ki67 in tumors from C57BL/6 mice intracranially implanted with CT2A cells expressing shC and shTfpi2. Scale bar, 50 mm. n=3 biological replicates. w,x, Representative (w) and quantification (x) of immunofluorescence staining of CC3 in tumors from C57BL/6 mice intracranially implanted with CT2A cells expressing shC and shTfpi2. Scale bar, 50 mm. n=3 biological replicates. Data from multiple replicates are presented as mean. Error bars indicate mean±SEM. One-way ANOVA test.

FIG. 11A-P. TFPI2 regulates GSC proliferation and apoptosis via JNK and STAT3 pathways. a,b, Representative images (a) and quantification (b) of proliferation in GSC23 expressing control and TFPI2 overexpression (OE) plasmid treated with or without JNK inhibitor JNK-IN-8 (10 nM). n=3 biological replicates. c,d, Representative images (c) and quantification (d) of proliferation in GSC262 expressing control and TFPI2 OE plasmid treated with or without JNK-IN-8 (10 nM). n=3 biological replicates. e,f, Representative images (e) and quantification (f) of apoptosis in GSC23 expressing control and TFPI2 OE plasmid treated with or without JNK-IN-8 (10 nM). n=3 biological replicates. g,h, Representative images (g) and quantification (h) of apoptosis in GSC262 expressing control and TFPI2 OE plasmid treated with or without JNK-IN-8 (10 nM). n=3 biological replicates. i,j, Representative images (i) and quantification (j) of proliferation in GSC23 expressing control and TFPI2 OE plasmid treated with or without STAT3 inhibitor WP1066 (20 nM). n=3 biological replicates. k,l, Representative images (k) and quantification (l) of proliferation in GSC262 expressing control and TFPI2 OE plasmid treated with or without WP1066 (20 nM). n=3 biological replicates. m,n, Representative images (m) and quantification (n) of apoptosis in GSC23 expressing control and TFPI2 OE plasmid treated with or without WP1066 (20 nM). n=3 biological replicates. o,p, Representative images (o) and quantification (p) of apoptosis in GSC262 expressing control and TFPI2 OE plasmid treated with or without WP1066 (20 nM). n=3 biological replicates. Data from multiple replicates are presented as mean. Error bars indicate mean±SEM. One-way ANOVA test.

FIG. 12A-K. TFPI2 promotes microglia migration through activation of STAT6 signaling. a, Amplification pattern of TFPI2 and CLOCK in GBM and low-grade glioma (LGG) patient tumors. b, GSEA analysis shows the top 10 enriched hallmark pathways in TFPI2-high GBM patient tumors compared to TFPI2-low tumors. Blue highlighted pathways relate to immune response. c, Representative images of transwell migration of PrhMG following stimulation with conditioned media (CM) from GSC262 or GSC23 cells expressing control and three independent TFPI2 overexpression (OE) plasmid clones. Scale bar, 400 mm. d, Representative images of transwell migration of PrhMG, HMC3 and SIM-A9 microglia following stimulation with CM from GSC272 cells (for PrhMG and HMC3) or QPP7 cells (for SIM-A9) expressing control shRNA (shC) and TFPI2 shRNAs (shTFPI2). Scale bars, 400 mm for PrhMG; 200 mm for HMC3 and SIM-A9. e,f, Representative images (e) and quantification (f) of flow cytometry analysis for the percentage of CD45^{low}CD11b⁺ and CD45^{high}CD11b⁺ cells in size matched tumors from C57BL/6 mice intracranially implanted with CT2A cells expressing shC and shTfpi2. n=3-4 biological replicates. g,h, Representative images (g) and quantification (h) of flow cytometry analysis for the percentage of CD45^{high}CD11b⁺CD68⁺ macrophages in size matched tumors from C57BL/6 mice intracranially implanted with CT2A cells expressing shC and shTfpi2. n=3-4 biological replicates. i, Correlation analysis between microglia signature and STAT6, AKT1, AKT2 or AKT3 in TCGA GBM dataset. R and P values are shown. Pearson test. j, Representative images of transwell migration of mouse primary microglia isolated from wild-type (WT) and STAT6 knockout (KO) mice treated with or without TFPI2 recombinant protein (20 ng/ml). Scale bar, 200 mm. k, Representative images of transwell migration of PrhMG, HMC3 and SIM-A9 microglia following stimulation with TFPI2 recombinant protein (20 ng/ml) in the presence or absence of STAT6 inhibitor AS1517499 at indicated concentrations. Scale bars, 400 mm for PrhMG; 200 mm for HMC3 and SIM-A9. Data from multiple replicates are presented as mean. Error bars indicate mean±SEM. One-way ANOVA test.

FIG. 13A-S. TFPI2-STAT6 axis is essential for microglia immunosuppressive polarization. a, Representative of flow cytometry analysis for CD206 in PrhMG treated with conditioned media (CM) from GSC23 cells expressing control and TFPI2 overexpression (OE) plasmid. n=3 biological replicates. b, Representative of flow cytometry analysis for CD206 in HMC3 microglia treated with CM from GSC272 expressing shRNA control (shC) and TFPI2 shRNAs (shTFPI2). c, Representative of flow cytometry analysis for CD206 in SIM-A9 microglia treated with CM from QPP7 GSCs expressing shC and shTfpi2 with or without reexpression of shRNA-resistant TFPI2 cDNA (OE). n=3 biological replicates. d,e, Representative images (d) and quantification (e) of flow cytometry analysis for the percentage of CD45^{hi}CD11b⁺CD68⁺CD206⁺ macrophages in size matched tumors from C57BL/6 mice intracranially implanted with CT2A cells expressing shC and shTfpi2. n=3-4 biological replicates. f,g, Immunofluorescence (f) and quantification (g) of CX3CR1⁺CD206⁺ cells in tumors from C57BL/6 mice intracranially implanted with CT2A cells expressing shC and shTfpi2. Scale bar, 25 □m. n=3 biological replicates. h,i, Immunofluorescence (h) and quantification (i) of CX3CR1⁺CD163⁺ cells in tumors from C57BL/6 mice intracranially implanted with CT2A cells expressing shC and shTfpi2. Scale bar, 25 □m. n=6 biological replicates. j,k, Immunofluorescence (j) and quantification (k) of CX3CR1⁺CD206⁺ cells in tumors from nude mice intracranially implanted with GSC272 cells expressing shC and shTFPI2. Scale bar, 25 Dim. n=6 biological replicates. l,m, Immunofluorescence (l) and quantification (m) of CX3CR1⁺CD163⁺ cells in tumors from nude mice intracranially implanted with GSC272 cells expressing shC and shTFPI2. Scale bar, 25 Dim. n=6 biological replicates. n, Representative of CD206 positive microglia isolated from wild-type (WT) and STAT6 knockout (KO) mice and treated with or without TFPI2 recombinant protein (20 ng/ml). o,p, Representative of flow cytometry analysis for CD206 (o) and ARG1 (p) in SIM-A9 microglia treated with TFPI2 recombinant protein (20 ng/ml) in the presence or absence of STAT6 inhibitor (STAT6i) AS1517499 (25 nM). q,r, Representative of flow cytometry analysis for CD206 (q) and ARG1 (r) in HMC3 microglia treated with TFPI2 recombinant protein (20 ng/ml) in the presence or absence of STAT6i AS1517499 (25 nM). s, Relative mRNA expression of IL6, TNFA, LFNG, and HLA-DR in HMC3 microglia treated with TFPI2 recombinant protein (20 ng/ml) in the presence or absence of STAT6i AS1517499 (25 nM). n=3 biological replicates. Data from multiple replicates are presented as mean. Error bars indicate mean±SEM. One-way ANOVA test.

FIG. 14A-T. CD51 is a receptor for TFPI2 activities on microglia. a, CD51 expression level in GBM/GSC cells (CD45⁻ cells), microglia (MG), macrophage (MDM), neutrophils, CD4⁺ T cells, and CD8⁺ T cells in human GBM sample from the TIME dataset. b, CD51 expression in different cell populations in mouse CT2A tumor tissues. c,d, Immunoprecipitation with TFPI2 antibody (TFPI2 Ab) and immunoblotting for the interaction between TFPI2 and CD51 in SIM-A9 (c) and HMC3 (d) microglia. e,f, Calcium mobilization triggered by TFPI2 recombinant protein (20 ng/ml) in HMC3 microglia (e) and PrhMG (f) treated with or without CD51 inhibitor MK-0429 at indicated concentrations. g, Immunoblots for CD51 in cell lysates of SIM-A9 microglia expressing shRNA control (shC) and Itgav shRNAs (shItgav). h, Immunoblots for P-PLCγ1, PLCγ1, P-PKCz, and PKCz in cell lysates of SIM-A9 microglia expressing shC and shItgav treated with or without TFPI2 recombinant protein (20 ng/ml). i, Immunoblots for P-STAT6 and STAT6 in cell lysates from SIM-A9 microglia and PrhMG treated with TFPI2 recombinant protein (20 ng/ml) and MK-0429 (50 nM). j, Representative images of relative migration of PrhMG, human HMC3, and mouse SIM-A9 microglia treated with or without TFPI2 recombinant protein (20 ng/ml) in the presence or absence of MK-0429 at indicated concentrations. Scale bars, 200 mm for HMC3 and SIM-A9, and 400 mm for PrhMG. k,l, Representative (k) and quantification (l) of flow cytometry analysis for the percentage of CD206⁺ SIM-A9 microglia treated with or without TFPI2 recombinant protein (20 ng/ml) and MK-0429 (50 nM). n=3 biological replicates. m,n, Representative (m) and quantification (n) of flow cytometry analysis for the percentage of ARG1⁺ SIM-A9 microglia treated with or without TFPI2 recombinant protein (20 ng/ml) and MK-0429 (50 nM). n=3 biological replicates. o,p, Representative (o) and quantification (p) of flow cytometry analysis for the percentage of CD206⁺ HMC3 microglia treated with or without TFPI2 recombinant protein (20 ng/ml) and MK-0429 (50 nM). n=3 biological replicates. q,r, Representative (q) and quantification (r) of flow cytometry analysis for the percentage of ARG1⁺ HMC3 microglia treated with or without TFPI2 recombinant protein (20 ng/ml) and MK-0429 (50 nM). n=3 biological replicates. s,t, Representative of flow cytometry analysis for the percentage of CD206⁺ (s) and ARG1⁺ (t) PrhMG treated with or without TFPI2 recombinant protein (20 ng/ml) and MK-0429 (50 nM). Data from multiple replicates are presented as mean. Error bars indicate mean±SEM. One-way ANOVA test.

FIG. 15A-P. TFPI2-STAT6 signaling mediates microglia-induced T cell suppression. a,b, Survival curves of nude mice implanted with QPP7 cells (2×10⁴ cells/mouse; a) or CT2A cells (2×10⁴ cells/mouse; b) expressing control shRNA (shC) and Tfpi2 shRNAs (shTfpi2). n=8 biological replicates. c,d, Immunofluorescence (c) and quantification (d) of CD8 in size matched tumors from C57BL/6 mice intracranially implanted with CT2A cells expressing shC and shTfpi2. Scale bar, 50 Dim. n=3 biological replicates. e,f, Immunofluorescence (e) and quantification (f) of CD4 in size matched tumors from C57BL/6 mice intracranially implanted with QPP7 GSCs expressing shC and shTfpi2. Scale bar, 50 □m. n=3 biological replicates. g, Representative of flow cytometry analysis for CD45⁺CD3⁺CD8⁺PD1^{high} and CD45⁺CD3⁺CD4⁺PD1^{high} T cells in the spleens of C57BL/6 mice bearing size matched CT2A tumors expressing shC and shTfpi2. h, Representative of flow cytometry analysis for IFNγ⁺CD8⁺ T cells cocultured with SIM-A9 mouse microglia treated with TFPI2 recombinant protein (20 ng/ml) in the presence or absence of STAT6 inhibitor (STAT6i) AS1517499 (25 nM). i, Representative of flow cytometry analysis for fixable viability dye-labeled JURKAT T cells cocultured with PrhMG treated with TFPI2 recombinant protein (20 ng/ml) in the presence or absence of STAT6i AS1517499 (25 nM). Percentage of live cells and dead cells are indicated. j,k, Representative (j) and quantification (k) of flow cytometry analysis for the percentage of HLA-DR⁺ cells out of total JURKAT T cells cocultured with PrhMG treated with TFPI2 recombinant protein (20 ng/ml) in the presence or absence of STAT6i AS1517499 (25 nM). n=3 biological replicates. l, Representative of flow cytometry analysis for CD69⁺ JURKAT T cells cocultured with PrhMG treated with TFPI2 recombinant protein (20 ng/ml) in the presence or absence of STAT6i AS1517499 (25 nM). m, Representative of flow cytometry analysis for IFNγ⁺ JURKAT T cells cocultured with PrhMG treated with TFPI2 recombinant protein (20 ng/ml) in the presence or absence of STAT6i AS1517499 (25 nM). n, CT2A cancer cell cytotoxicity induced by activated mouse primary CD8⁺ T cells (the ratios between CD8⁺ T cells and CT2A cells are indicated) cocultured with SIM-A9 microglia treated with TFPI2 recombinant protein (20 ng/ml) in the presence or absence of STAT6i AS1517499 (25 nM). o, Correlation analysis between CD274 (PD-L1) and TFPI2, STAT6, or ITGAV (CD51) in TCGA GBM dataset. R and P values are shown. Pearson test. p, Correlation analysis between PDCD1LG2 (PD-L2) and TFPI2, STAT6, or ITGAV (CD51) in TCGA GBM dataset. R and P values are shown. Pearson test. Data from multiple replicates are presented as mean. Error bars indicate mean±SEM. One-way ANOVA test. In (a,b), log-rank test.

FIG. 16A-F. TFPI2 correlates positively with proliferation and negatively with apoptosis in GBM patient tumors. a, Representative images show low, medium, and high expression of TFPI2, Ki67, and cleaved caspase 3 (CC3) in human GBM tumor samples based on immunohistochemistry staining. Scale bar, 100 mm. b,c, Quantification of immunohistochemistry staining showing strong positive correlation between TFPI2 and Ki67 (b) and negative correlation between TFPI2 and CC3 (c) in human GBM tumor samples (n=24). R and P values are shown. Pearson test. d-f, The plasma TFPI2 level in newly diagnosed and recurrent (d), female and male (e), as well as old (>65) and young (<65) GBM patients (f). Error bars indicate mean±SEM. Student's t-test.

FIG. 17A-B. GSEA for stemness signature (a) and microglia signature (b) in TFPI2-amplified (amp) patient tumors compared with TFPI2-non amplified tumors from TCGA GBM dataset. NES and FDR values are shown.

FIG. 18A-I. a, Immunoblots for TFPI2, CD133, and SOX2 in cell lysates from human GSC2 expressing shRNA control (shC) and TFPI2 shRNAs (shTFPI2). b,c, Representative images (b) and quantification (c) of tumorspheres in GSC2 expressing shC and shTFPI2. n=12 biological replicates. Scale bar, 200 μm. d,e, Representative images (d) and quantification (e) of tumor from 32 days post-orthotopic injection of QPP7 cells (2×10⁴ cells/mouse) expressing shC and shTfpi2. n=6 biological replicates. f,g, Representative images (f) and quantification (g) of tumor from 22 days post-orthotopic injection of CT2A cells (2×10⁴ cells/mouse) expressing shC and shTfpi2. n=6 biological replicates. h,i, Representative images (h) and quantification (i) of tumor from 30 days post-orthotopic injection of GSC272 cells (2×10⁵ cells/mouse) expressing shC and shTFPI2. n=7-8 biological replicates. Error bars indicate mean±SEM. One-way ANOVA test.

FIG. 19A-B. a, Immunoblots for P-STAT3 and STAT3 in cell lysates of GSC23 and GSC262 cells treated with TFPI2 recombinant protein (20 ng/ml) in the presence or absence of JNK inhibitor JNK-IN-8 (10 nM). b, Immunoblots for P-JNK and JNK in cell lysates of GSC23 and GSC262 cells treated with TFPI2 recombinant protein (20 ng/ml) in the presence or absence of STAT3 inhibitor WP1066 (20 nM).

FIG. 20A-B. Representative images (a) and quantification (b) of transwell migration of SIM-A9 microglia following stimulation with conditioned media (CM) from QPP7 cells expressing control shRNA (shC) and Tfpi2 shRNAs (shTfpi2) with or without TFPI2 overexpression (OE). n=3 biological replicates. Scale bars, 200 μm. Data from multiple replicates are presented as mean. Error bars indicate mean±SEM. One-way ANOVA test.

FIG. 21. Representative images of scratch assay on HMC3 microglia treated with TFPI2 recombinant protein (20 ng/ml) in the presence or absence of STAT6 inhibitor AS1517499 (25 nM) for indicated time points. Scale bars, 200 μm.

FIG. 22A-F. a,b, Immunofluorescence (a) and quantification (b) of CX3CR1 (green) and ARG1 (red) double positive cells in QPP7 tumors expressing control shRNA (shC) and Tfpi2 shRNAs (shTfpi2). Scale bar, 25 μm. n=4 biological replicates. c,d, Immunofluorescence (c) and quantification (d) of CX3CR1 (green) and ARG1 (red) double positive cells in CT2A tumors expressing shC and shTfpi2. Scale bar, 25 μm. n=5 biological replicates. e,f, Immunofluorescence (e) and quantification (f) of CX3CR1 (green) and ARG1 (red) double positive cells in GSC272 tumors expressing shC and shTFPI2. Scale bar, 25 μm. n=6 biological replicates. Data from multiple replicates are presented as mean. Error bars indicate mean±SEM. one-way ANOVA test.

FIG. 23A-C. a, quantification of incucyte live-cell migration of HMC3 microglia treated with TFPI2 recombinant protein (20 ng/ml) in the presence or absence of CD51 inhibitor MK0429 (50 nM). b,c, Representative images (b) and quantification (c) of scratch assay on HMC3 microglia treated with TFPI2 recombinant protein (20 ng/ml) in the presence or absence of CD51 inhibitor MK0429 (50 nM) for indicated time points. Scale bars, 200 μm.

FIG. 24A-B. Illustration shows a proposed mechanism for the TFPI2 and its associated signaling axis in regulating self-renewal of glioma stem cells (GSCs) and GSC-microglia symbiosis in GBM. TFPI2 is amplified in GBM and essential for tumor progression. Upon secreting from GSCs, TFPI2 promotes GSC self-renewal by activating the JNK-STAT3 signaling pathway. Secreted TFPI2 binds to CD51 on microglia to promote microglia infiltration and immunosuppressive polarization via activation of STAT6 signaling. Consequently, these activated microglia inhibit the infiltration and activation of T cells and damage their tumor killing potential. b, Inhibition of TFPI2-CD51-STAT6 signaling axis impairs tumor progression by reducing immunosuppressive microglia and activating T cell-mediated anti-tumor immunity and synergizes with anti-PD1 therapy in GBM. This image was created with BioRender.com.

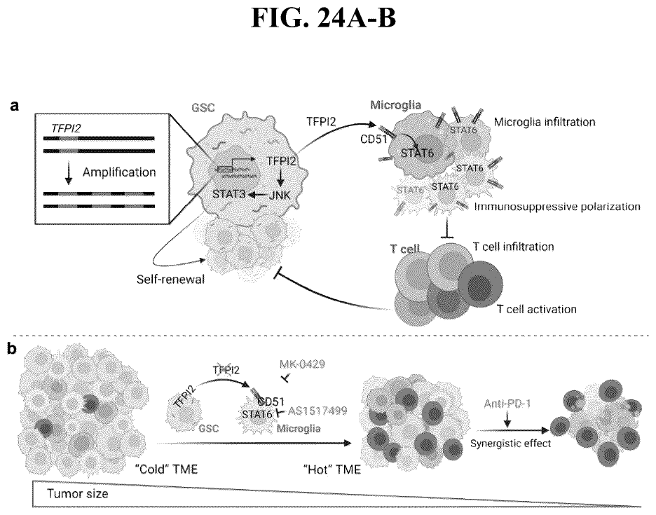

FIG. 25. Gating strategy for flow cytometry after Apotracker and propidium iodide (PI) staining. During flow cytometer acquisition and data analysis, glioma stem cell (GSC) samples were sequentially gated (1) for morphology by plotting forward scatter area (FSC-A) and side scatter area (SSC-A); (2) for singlets by plotting FSC-A and forward scatter height (FSC-H); and (3) for apoptosis by plotting Apotracker (FITC) and PI using quad gates.

FIG. 26. Gating strategy for flow cytometry of cells from brain tumor tissues. During flow cytometer acquisition and data analysis, samples were sequentially gated (1) for morphology by plotting FSC-A and SSC-A; (2) for singlets by plotting FSC-A and FSC-H; and (3) for live cells by plotting fixable viability dye (APC Cy7) and SSC-A; (4) for macrophage and microglia by plotting CD45 and CD11b; (5) for more specific macrophage population by plotting CD68 and SSC-A; (6) for more specific microglia population by plotting CX3CR1 and SSC-A. (CD45hiCD11b⁺CD68⁺) or microglia (CD45loCD11b⁺CX3CR1⁺) were finally analyzed for immunosuppressive marker CD206.

FIG. 27. Gating strategy for flow cytometry of in vitro cultured microglia (SIM-A9, PrhMG, and HMC3). During flow cytometer acquisition and data analysis, samples were sequentially gated (1) for morphology by plotting FSC-A and SSC-A; (2) for singlets by plotting FSC-A and FSC-H; and (3) for live cells by plotting fixable viability dye (APC Cy7) and SSC-A. Viable cells were finally analyzed for CD206 and ARG1.

FIG. 28. Gating strategy for flow cytometry of splenic lymphocytes from tumor-bearing mice. During flow cytometer acquisition and data analysis, samples were sequentially gated (1) for morphology by plotting FSC-A and SSC-A; (2) for singlets by plotting FSC-A and FSC-H; and (3) for live cells by plotting fixable viability dye (APC Cy7) and SSC-A; (4) for total T cell by plotting CD45 and CD3; (5) for CD8 T cell and CD4 T cell by plotting CD8 and CD4, respectively. CD4 T cell (CD45⁺CD3⁺CD4⁺) or CD8 T cell (CD45⁺CD3⁺CD8⁺) were finally analyzed for exhaustion marker PD1.

FIG. 29. Gating strategy for flow cytometry of mouse primary CD8⁺ T cells following co-culture with mouse SIM-A9 microglia. During flow cytometer acquisition and data analysis, samples were sequentially gated (1) for morphology by plotting FSC-A and SSC-A; (2) for singlets by plotting FSC-A and FSC-H; and (3) for CD8⁺ T cells by plotting CD8 and SSC-A. CD8⁺ T cells were finally analyzed for IFNγ and CD69.

FIG. 30. Gating strategy for flow cytometry of human JURKAT cells following co-culture with human primary microglia (PrhMG). During flow cytometer acquisition and data analysis, samples were sequentially gated (1) for morphology by plotting FSC-A and SSC-A; (2) for singlets by plotting FSC-A and FSC-H; and (3) for live cells by plotting fixable viability dye (APC Cy7) and SSC-A; (3) for JURKAT cells by plotting CD11b and SSC-A. CD11b⁻ JURKAT cells were finally analyzed for HLA-DR, IFNγ and CD69.

FIG. 31. Treatment of CD51 inhibitor Cilengitide significantly extended the survival of 005 GSC and CT2A tumor-implanted mice. Cilengitide enhanced the efficacy of anti-PD1 therapy on GBM mouse models.

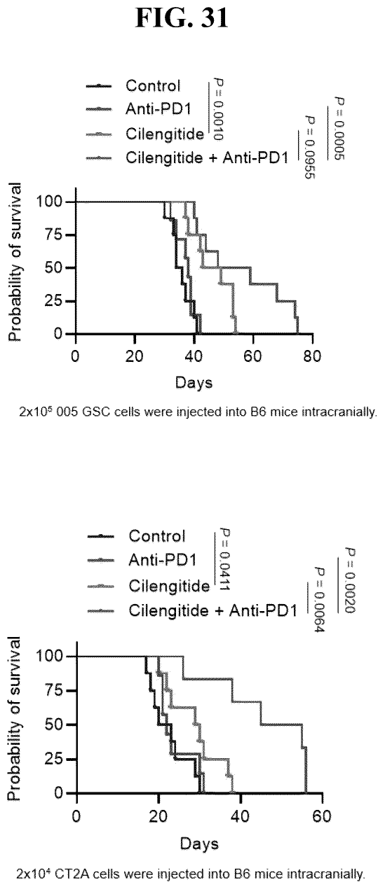

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a” “an” and “the” include plural reference unless the context clearly dictates otherwise. Thus, for example, reference to “a TFPI2 inhibitor” is a reference to one or more TFPI2 inhibitors and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “pharmaceutically acceptable carrier” refers to non-toxic solid, semisolid, or liquid filler, diluent, encapsulating material, formulation auxiliary, excipient, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., the copolymers described herein) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “antibody” refers to a whole antibody molecule or a fragment thereof (e.g., fragments such as Fab, Fab′, and F(ab′)₂), unless specified otherwise; an antibody may be polyclonal or monoclonal antibody, a chimeric antibody, a humanized antibody, a human antibody, etc.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

### DETAILED DESCRIPTION

Provided herein are inhibitors of the TFPI2-CD51-STAT6 signaling axis and methods for the treatment of cancer (e.g., glioblastoma) therewith. In particular, inhibitors of expression or activity of components of the TFPI2-CD51-STAT6 signaling axis are provided, with or without an immune checkpoint inhibitor, for the treatment of glioblastoma.

Glioblastoma (GBM) tumors contain multiple cell populations, including self-renewing glioblastoma stem cells (GSCs) and immunosuppressive microglia. Experiments conducted during development of embodiments herein identified Kunitz-type protease inhibitor TFPI2 as a critical factor connecting these cell populations and their associated GBM hallmarks of stemness and immunosuppression. TFPI2 promotes GSC self-renewal and tumor growth via activation of the c-Jun N-terminal kinase (JNK)-signal transducer and activator of transcription 3 (STAT3) pathway. Secreted TFPI2 interacts with its functional receptor CD51 on microglia to trigger the infiltration and immunosuppressive polarization of microglia through activation of STAT6 signaling. Inhibition of the TFPI2-CD51-STAT6 signaling axis activates T cells and synergizes with anti-PD1 therapy in GBM mouse models. In human GBM, TFPI2 correlates positively with stemness, microglia abundance, immunosuppression, and poor prognosis. Experiments conducted during development of embodiments herein reveal unexpected roles of TFPI2 in GBM biology and supports therapeutic targeting of TFPI2 and/or the TFPI2-CD51-STAT6 signaling axis as an effective strategy for this deadly disease.

Experiments conducted during development of embodiments herein identified tissue factor pathway inhibitor 2 (TFPI2) has been identified as a key effector of the symbiotic interaction between GSCs and TAMs. TFPI2 is a member of the Kunitz-type serine proteinase inhibitor family. Despite its name and structural similarity to TFPI1, a potent inhibitor for factors Xa and VIIa/TF (tissue factor) complex in the extrinsic pathway of blood coagulation, TFPI2 has little inhibitory effect on TFs (Refs. 13-14; incorporated by reference in their entireties). Instead, TFPI2 is recognized as a critical factor that can inhibit extracellular matrix (ECM) degradation via suppressing plasmin-dependent activation of pro-matrix metalloproteinase 1 (MMP1) and pro-MMP13. Since ECM degradation is important for tumor growth and metastasis (ref. 15; incorporated by reference in its entirety), TFPI2 has been considered as a tumor suppressor (Refs. 16-17; incorporated by reference in its entirety). However, contrasting results indicate a pro-tumor activity of TFPI2 in multiple cancers, including hepatocellular carcinoma (Ref. 18; incorporated by reference in its entirety), melanoma (Refs. 19-20; incorporated by reference in their entireties), and ovarian clear cell carcinoma (Ref. 21; incorporated by reference in its entirety). These findings suggest that the role of TFPI2 in tumor biology may be context and cancer type dependent. In GBM, TFPI2 has been shown to inhibit the growth, survival, migration, and invasion of differentiated GBM cells (Refs. 22-24; incorporated by reference in their entireties).

Experiments conducted during development of embodiments herein identified an unexpected function for TFPI2 in supporting maintenance of stemness and tumor growth via activation of the c-Jun N-terminal kinase (JNK)-signal transducer and activator of transcription 3 (STAT3) pathway in GSCs. Moreover, secreted TFPI2 contributes to an immunosuppressive TME by promoting microglia infiltration and polarization via activation of CD51 (also known as integrin alpha V, ITGAV)-STAT6 signaling pathway in microglia. Inhibition of the TFPI2-CD51-STAT6 pathway in GBM mouse models activates antitumor immunity that extends the survival of tumor-bearing animal subjects. This anti-tumor immune activation can be coupled with immune checkpoint inhibitor (ICI) to further extend survival. In combination with results from analysis of patient tumor and plasma samples, experiments conducted during development of embodiments herein demonstrate that TFPI2 as a therapeutic target for treating GBM.

In some embodiments, compositions and methods described herein employ a TFPI2 inhibitor or a composition/method for the inhibition of TFPI2 activity or expression.

In some embodiments, compositions and methods described herein employ a CD51 inhibitor or a composition/method for the inhibition of CD51 activity or expression.

In some embodiments, compositions and methods described herein employ a STAT6 inhibitor or a composition/method for the inhibition of STAT6 activity or expression.

In some embodiments, provided herein are inhibitors of TFPI2, CD51, and/or STAT6 activity. In some embodiments, an inhibitor is a small molecule, peptide, antibody, antibody fragment, a genome editing agent, etc. that upon administration to a cell, reduces the activity of TFPI2, CD51, and/or STAT6.

In some embodiments, a TFPI2, CD51, and/or STAT6 inhibitor is an antibody or antibody fragment. In some embodiments, a TFPI2, CD51, and/or STAT6 inhibitor is an antibody or antibody fragment that binds to TFPI2, CD51, or STAT6 and reduces TFPI2, CD51, and/or STAT6 activity. In some embodiments, an anti-TFPI2, anti-CD51, or anti-STAT6 antibody or antibody fragment is provided that reduced the activity of TFPI2, CD51, or STAT6 and/or reduces binding of TFPI2, CD51, or STAT6 to a ligand thereof. In some embodiments, an anti-CD51 antibody is selected from Abituzumab, Anti-ITGAV-DM4 ADC (ADC-W-109), and Anti-ITGAV (clone MF-T)-SPDB-DM4 ADC (ADC-W-422). An anti-STAT6 is selected from Phospho-STAT6-Y641 Antibody Blocking Peptide (BP3270a) and Eblasakimab.

In some embodiments, a TFPI2 inhibitor is a small molecule, protein (e.g., antibody) or peptide that binds to TFPI2 and inhibits its activity.

In some embodiments, a CD51 inhibitor is a small molecule, protein (e.g., antibody) or peptide that binds to TFPI2 and inhibits its activity. In some embodiments, a CD51 inhibitor is selected from cilengitide, MK-0429, Cyclo(-RGDfK), Cyclo(RGDyK) trifluoroacetate, Cyclo(-RGDfK) TFA, Cyclo RGDfC, HSDVHK-NH2, Cyclo(Arg-Gly-Asp-D-Phe-Cys) TFA, Echistatin, Nesvategrast, Cyclo(RGDyK), JNJ-26076713, Bexotegrast, EMD527040, and abituzumab.

In some embodiments, a STAT6 inhibitor is a small molecule, protein (e.g., antibody) or peptide that binds to STAT6 and inhibits its activity. STAT6 activity has been inhibited by small molecules (Nagashima, et al. Bioorg. Med. Chem. 2007, 15, 1044-1055; Nagashima, et al. Bioorg. Med. Chem. 2008, 16, 6509-6521.; Nagashima, et al. Bioorg. Med. Chem. 2009, 17, 6926-6936.; Ohga, et al. Eur. J. Pharmacol. 2008, 590, 409-416.; Chiba, et al. Am. J. Respir. Cell Mol. Biol. 2009, 41, 516-524.; incorporated by reference in their entireties) and antibodies (Walsh, G. M. Expert Opin. Emerg. Drugs 2012, 17, 37-42.; and Blease. Curr. Opin. Investig. Drugs 2008, 9, 1180-1184.; incorporated by reference in their entireties). STAT6 expression has been inhibited by, for example, siRNA (Darcan-Nicolaisen, Y. et al. J. Immunol. 2009, 182, 7501-7508; incorporated by reference in its entirety). Examples of STAT6 inhibitors include AS 1571499, AS 1617612, AS 1810722, (R)-76, (R)-84, and those described in, for example, U.S. Pub. No. 2016/0145279; incorporated by reference in its entirety. In some embodiments, a STAT6 inhibitor is selected from AS 1571499, AS 1617612, AS 1810722, (R)-76, (R)-84, STAT6-IN-1, STAT6-IN-2, STAT6-IN-3, PM-43I, PM-81I, and Picroside I.

In some embodiments, provided herein are inhibitors of TFPI2, CD51, and/or STAT6 expression. In particular embodiments, an inhibitor TFPI2, CD51, and/or STAT6 expression is a nucleic acid-based inhibitor. In some embodiments, the inhibitor is a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, a transcription activator-like (TAL) effector (TALE) nuclease, etc.

In some embodiments, the inhibitor TFPI2, CD51, and/or STAT6 expression is a small interfering RNA (siRNA), also known as short interfering RNA or silencing RNA. In some embodiments, an siRNA is an 18 to 30 nucleotide, preferably 19 to 25 nucleotide, most preferred 21 to 23 nucleotide or even more preferably 21 nucleotide-long double-stranded RNA molecule. siRNA is involved in the RNA interference (RNAi) pathway where the siRNA interferes with the expression of a specific gene (e.g., TFPI2, CD51, or STAT6). siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., TFPI2, CD51, or STAT6). Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit TFPI2, CD51, or STAT6 is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP). In some embodiments, provided herein are siRNA molecules that target and inhibit the expression (e.g., knock down) of TFPI2, CD51, or STAT6.

A short hairpin RNA (shRNA) is a sequence of RNA that makes a tight hairpin turn that can be used to silence gene expression (e.g., TFPI2, CD51, or STAT6) via RNA interference. In some embodiments, shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). The RISC binds to and cleaves RNAs which match the siRNA that is bound to (e.g., comprising the sequence of TFPI2, CD51, or STAT6). In some embodiments, si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knock down) of TFPI2, CD51, or STAT6.

Further molecules effecting RNAi (and useful herein for the inhibition of expression of TFPI2, CD51, or STAT6) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of TFPI2, CD51, or STAT6 after introduction into target cells. In some embodiments, provided herein are miRNA molecules that target and inhibit the expression (e.g., knock down) of the TFPI2, CD51, or STAT6.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., TFPI2, CD51, or STAT6) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of TFPI2, CD51, or STAT6.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA (e.g., a portion of TFPI2, CD51, or STAT6), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors of the TFPI2, CD51, or STAT6.

In some embodiments, TFPI2, CD51, or STAT6 is inhibited by modifying the TFPI2, CD51, or STAT6 sequence in target cells. In some embodiments, the alteration of TFPI2, CD51, or STAT6 is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within TFPI2, CD51, or STAT6) and fixed tracrRNA) are introduced into the cell. In general, target sites at the 5′ end of the gRNA target the Cas nuclease to the target site using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within the TFPI2, CD51, or STAT6 gene). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site (e.g., within the TFPI2, CD51, or STAT6 gene). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of TFPI2, CD51, or STAT6). In some embodiments, the CRISPR system is used to alter TFPI2, CD51, or STAT6, inhibit expression of TFPI2, CD51, or STAT6, and/or to inactivate the expression product of the TFPI2, CD51, or STAT6.

The term “antisense nucleic acid molecule” or “antisense oligonucleotide” as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901). In some embodiments, provided herein are antisense oligonucleotides capable of inhibiting expression of TFPI2, CD51, or STAT6 when administered to cell or subject. In some embodiments, the antisense oligonucleotides are antisense DNA- and/or RNA-oligonucleotides. In some embodiments, provided herein are modified antisense oligonucleotides, such as, antisense 2′-O-methyl oligo-ribonucleotides, antisense oligonucleotides containing phosphorothiaote linkages, antisense oligonucleotides containing Locked Nucleic Acid LNA(R) bases, morpholino antisense oligonucleotides, PPAR-gamma agonists, antagomirs. In some embodiments, ASOs comprise Locked Nucleic Acid (LNA) or 2′-methoxyethyl (MOE) modifications (internucleotide linkages are phosphorothioates interspersed with phosphodiesters, and all cytosine residues are 5′-methylcytosines).

In some embodiments, an inhibitor of the TFPI2-CD51-STAT6 signaling axis is administered for the treatment of a cancer. Certain embodiments herein are directed to administration of an inhibitor of the TFPI2-CD51-STAT6 signaling axis to a subject with cancer, in remission from cancer, or at elevated risk of cancer. In some embodiments, an inhibitor of the TFPI2-CD51-STAT6 signaling axis is administered as part of therapeutic or prophylactic regimen for the treatment or prevention of acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In particular embodiments, an inhibitor of the TFPI2-CD51-STAT6 signaling axis is administered for the treatment of glioblastoma (e.g., primary glioblastoma, secondary glioblastoma, etc.).

In some embodiments, an inhibitor of the TFPI2-CD51-STAT6 signaling axis is co-administered with one or more additional agents (e.g., for the treatment of cancer).

In some embodiments, an inhibitor of the TFPI2-CD51-STAT6 signaling axis is co-administered with an immune checkpoint inhibitor. Immune checkpoint inhibitors are a class of drugs designed to enhance the body's natural immune response against cancer and other diseases by blocking certain molecules (checkpoints) that regulate immune responses. Immune checkpoint molecules are ac component of systems that helps maintain immune balance and prevent excessive immune activation. Cancers often exploit immune checkpoint pathways to evade detection and attack by the immune system. By inhibiting specific immune checkpoints, immune checkpoint inhibitors can reactivate the immune response, enabling immune cells to recognize and attack cancer cells more effectively. Immune checkpoint inhibitors work by targeting specific cell surface molecules on immune cells or cancer cells. These molecules are often ligands or receptors that, when engaged, suppress immune responses. By blocking the interactions between these molecules, checkpoint inhibitors can enhance immune responses against cancers. In some embodiments, an inhibitor of the TFPI2-CD51-STAT6 signaling axis is co-administered with an immune checkpoint inhibitor (e.g., antibody or fragment thereof) that targets an immune checkpoint molecule/protein selected from the group consisting of CTLA4, PD-1, PD-L1, PD-L2, A2AR, B7-H3, B7-H4, BTLA, KIR, LAG3, TIM-3, VISTA, or a ligand/receptor thereof. In some embodiments, an inhibitor of the TFPI2-CD51-STAT6 signaling axis is co-administered with an immune checkpoint inhibitor selected from nivolumab, pembrolizumab, atezolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

In some embodiments, an inhibitor of the TFPI2-CD51-STAT6 signaling axis (alone or co-administered with an immune checkpoint inhibitor) is co-administered along with administration of a chemotherapy agent. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

In some embodiments, an inhibitor of the TFPI2-CD51-STAT6 signaling axis (alone or co-administered with an immune checkpoint inhibitor) is co-administered with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with an inhibitor of the TFPI2-CD51-STAT6 signaling axis (alone or co-administered with an immune checkpoint inhibitor). Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, and MMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, medicaments which are administered in conjunction with an inhibitor of the TFPI2-CD51-STAT6 signaling axis (alone or co-administered with an immune checkpoint inhibitor) include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatories, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (-)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon. Exemplary therapeutic agents useful for a combination therapy with an inhibitor of the TFPI2-CD51-STAT6 signaling axis (alone or co-administered with an immune checkpoint inhibitor) include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Other suitable therapeutic agents for coadministration with an inhibitor of the TFPI2-CD51-STAT6 signaling axis (alone or co-administered with an immune checkpoint inhibitor) also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated for co-administration with an inhibitor of the TFPI2-CD51-STAT6 signaling axis (alone or co-administered with an immune checkpoint inhibitor) include diuretics, vasopressin, agents affecting the renal conservation of water, rennin, angiotensin, agents useful in the treatment of myocardial ischemia, anti-hypertensive agents, angiotensin converting enzyme inhibitors, 0-adrenergic receptor antagonists, agents for the treatment of hypercholesterolemia, and agents for the treatment of dyslipidemia.

Other therapeutic agents contemplated for co-administration with an inhibitor of the TFPI2-CD51-STAT6 signaling axis (alone or co-administered with an immune checkpoint inhibitor) include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, Mycobacterium avium complex disease, and leprosy, antifungal agents, antiviral agents including nonretroviral agents and antiretroviral agents.

Examples of therapeutic antibodies that can be combined with an inhibitor of the TFPI2-CD51-STAT6 signaling axis (alone or co-administered with an immune checkpoint inhibitor) include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

### EXPERIMENTAL

### Materials and Methods

### Cell Culture

HMC3 microglia, 293T and CT2A cells, SIM-A9 microglia, as well as SB28 and GL261 cells were cultured in Eagle's Minimum Essential Medium (ATCC, #30-2003), Dulbecco's Modified Eagle's Medium (DMEM; Gibco, #11995-065), DMEM-Ham's F12 medium (Gibco, #10565-018), and RPMI 1640 medium (RPMI; Gibco, #11875093), respectively, containing 10% fetal bovine serum (FBS, Fisher Scientific, #16140071) and 1:100 antibiotic-antimycotic (Gibco, #15140-122). For stemness maintenance, CT2A cells were cultured in neural stem cell (NSC) proliferation media (Millipore, #SCM005) containing 20 ng/ml epidermal growth factor (EGF; PeproTech, #AF-100-15) and basic fibroblast growth factor (bFGF; PeproTech, #100-18B). Similarly, human (GSC2, GSC17, GSC20, GSC23, GSC262, GSC272) and mouse (QPP7 and 005 GSC) GSCs were cultured in NSC proliferation media containing 20 ng/ml EGF and bFGF. Human GSCs were gifted by Dr. Frederick F. Lang from the Brain Tumor Center (The University of Texas MD Anderson Cancer Center). QPP7 and 005 GSCs were provided by Dr. Jian Hu (The University of Texas MD Anderson Cancer Center) and Dr. Samuel D. Rabkin (Massachusetts General Hospital), respectively. JURKAT cells were cultured in human plasma-like medium (Thermo Fisher Scientific, #A4899101) containing 10% FBS and 1:100 antibiotic-antimycotic. All cells were confirmed to be mycoplasma-free and were maintained at 37° C. and 5% CO₂. Conditioned media (CM) were collected from number-matched control, TFPI2 shRNA knockdown, or TFPI2-overexpressing cells after culturing for another 24 hrs in FBS- and growth factor-free culture medium.

### Isolation and Culture of Primary CD8⁺ T Cells and Microglia

Primary mouse CD8⁺ T cells were isolated from the spleens of C57BL/6 mice using a CD8⁺ T cell isolation kit (Miltenyi Biotec, #130-104-075) according to manufacturer's instructions. Isolated CD8⁺ T cells were cultured in RPMI 1640 media (Gibco, #22400-089) containing 10% FBS, 50 μM 2-Mercaptoethanol (Sigma, #M7522), and 1:100 antibiotic-antimycotic. Then, primary mouse CD8⁺ T cells were expanded by 30 U/ml mouse IL-2 (Miltenyi Biotec, #130-120-662) and activated by Dynabeads® mouse T-activator CD3/CD28 (Gibco, #11456D) at bead-to-cell ratio of 1:1 before analysis and co-culture studies.

Adult human primary microglia (PrhMG) were isolated (Ref. 38; incorporated by reference in its entirety). Fresh cortical tissues from frontal poles of postmortem human brains were harvested at autopsy. Brain tissues were rinsed with cold RPMI 1640 medium on ice. After removing meninges and visible blood vessels, blocks of cortex were minced and incubated in a 0.25% trypsin EDTA solution for 1 hr. The suspension was passed through 100 mm and 40 mm Nitex mesh filters and resuspended in microglia media (ScienCell Research Laboratories, #1901) with 5% FBS, 100 mg/ml antibiotic-antimycotic, 1.5 ml/500 ml Primocin (InvivoGen, #ant-pm-05), 1% microglia growth supplement (ScienCell, #1952), and 10 ng/ml granulocyte-macrophage colony-stimulating factor (PeproTech, #315-03). Cells were then labeled with CD11b magnetic beads and MACS method was used to select CD11b⁺ cells. After selection, cells were cultured in poly-D-lysine coated plates.

Brains of neonate mice were homogenized in 10 ml HBSS on ice. Brain-derived microglia were then purified using 30/70 Percoll (GE Healthcare, #17-0891-01) with gradient separation for 30 min at 4° C. The isolated microglia were cultured in poly-D-lysine coated plates with microglia media containing 5% FBS, 100 mg/ml antibiotic-antimycotic, 1.5 ml/500 ml Primocin, 1% microglia growth supplement, and 10 ng/ml granulocyte-macrophage colony-stimulating factor. The purity of microglia was determined by flow cytometry analysis, in which more than 90% of cells are CX3CR1 positive.

### Plasmids, Viral Transfections, and Cloning

shRNAs targeting human TFPI2, mouse Tfpi2, and mouse Itgav in the pLKO.1 vector (Sigma, #SHC001) were used. Lentiviral particles were generated (Ref. 61; incorporated by reference in its entirety). The following mouse and human shRNA sequences (TFPI2: #1 TRCN0000373908, #2 TRCN0000373822, #3 TRCN0000072725; Tfpi2: #2 TRCN0000271824, #3 TRCN0000271717; and Itgav: #2 TRCN0000066588, #5 TRCN0000066590, #6 TRCN0000066591) were selected following validation. Lentiviral particles were generated (Refs. 11-12, 61; incorporated by reference in their entireties). 8 mg of the shRNA and packaging plasmids (4 mg psPAX2 and 2 mg pMD2.G) were transfected using Lipofectamine 2000 (Invitrogen, #13778150) into 293T cells. Supernatant with lentiviral particles was collected at 48 and 72 hrs after transfection. GSCs or microglia were infected with lentiviral supernatant containing 10 mg/ml polybrene (Millipore, #TR1003-G) and then selected using 2 mg/ml puromycin (Millipore, #540411). Proteins were extracted to assess the expression of TFPI2 or CD51 by immunoblots. For rescue experiments, TFPI2 shRNA knockdown QPP7 cells were transfected with a human TFPI2 construct that is resistant to TFPI2 shRNAs.

CRISPR KO of TFPI2 in human GSC272 cells was produced from the transfection of sgRNA (GenScript, #SC1969) targeting the TFPI2 protein sequence (TTCTCCGTTACTACTACGAC) and Cas9 Nuclease (GenScript, #Z03621-0.5). 50 pmol of Cas9 Nuclease and 100 pmol of sgRNA were incubated in 125 ml of Opti-MEM media (Gibco, #31985088) for 10 mins at room temperature before combining with an additional 125 ml of Opti-MEM media containing Lipofectamine™ CRISPRMAX™ (Invitrogen, #CMAX00001) transfection reagent. GSC272 cells were incubated with the cocktail of sgRNA, Cas9 nuclease, and transfection reagent for 48 hrs. Following the incubation period, transfected GSC272 cells were expanded in culture, then taken for GFP tag cell-sorting using FACS ARIA 4-Laser Sorter. GFP⁺ cells were returned to normal cell culture conditions for expansion. CRISPR KO efficiency was verified using immunoblot to assess TFPI2 expression.

For overexpression, GSCs were transfected with a TFPI2 overexpression plasmid generated via a cloning methodology. A pCMV6-Entry vector containing the TFPI2 (Myc-DDK-tagged) sequence (Origene, #RC202760) and a pLenti-C-mGFP Lentiviral Gene Expression Vector (Origene, #PS100071) were digested using restriction enzymes corresponding to the open reading frames. Restriction enzymes Mlul (Promega, #R6381) and Sgfl (Promega, #R7103) were mixed with Restriction Digest Buffer C to enhance digestion efficiency. Following digestion, each mixture was incubated with antarctic phosphatase for 3 hrs to prevent self-ligation. T4 DNA ligase (Thermo Scientific, #B69) was included in a ligation mixture to induce the ligation of the TFPI2 insert into the pLenti-C-mGFP Lentiviral Gene Expression Vector. Ligation mixture was transformed into high-efficiency chemically competent E. coli cells (Thermo Scientific, #C737303), and recovered in LB broth (Fisher BioReagents, #BP9723). Post-recovery, LB broth containing E. coli transformants were plated on LB selection plates containing 34 μg/ml chloramphenicol (Fisher BioReagents, #BP904) for selection of clones containing the TFPI2 ORF in the pLent-C-mGFP Lentiviral Gene Expression Vector. After 16 hrs of incubation at 37° C., three colonies were picked from the selection plates for inoculation in LB Broth supplemented with 34 μg/ml chloramphenicol to maintain selection, and then further purified for plasmid DNA using a QIAprep Spin Miniprep Kit (Qiagen, #27106). Purified plasmids were then transfected into GSCs using lentiviral transfection methodology (Ref. 12; incorporated by reference in its entirety).

### Migration Assays

For transwell migration assay, HMC3 microglia, SIM-A9 microglia, and PrhMG were suspended in serum-free culture medium and seeded into transwell 24-well plates with permeable polycarbonate membrane inserts (5.0 μm, Corning, #07-200-149). GSC conditioned media (CM), or basal cell culture medium with TFPI2 recombinant protein (BioVision, #7481-10) were added to the remaining receiver wells. The STAT6 inhibitor AS151749 (Selleck Chemicals, #S8685) was used to study the role of STAT6 in microglia migration. The CD51 inhibitor MK-0429 (MedChemExpress, #HY-15102) was used to study the role of CD51 in TFPI2-induced microglia migration. After 10 hrs, the migrated microglia were fixed and stained with crystal violet (Sigma, #C-3886). The number of transferred cells was counted using ImageJ (NIH, Bethesda, ML).

The scratch-wound assay was performed (Ref. 74; incorporated by reference in its entirety). For tracking single cell movement, microglia were cultured in 96-well plates (Corning, #3599) with different treatments. Incucyte Live Cell Analysis System was used to record cell movement over time. The time-lapse images were reconstructed and analyzed using TrackMate (Refs. 75-76; incorporated by reference in its entirety). The speed of each individual cell was calculated by processing images with LoG detector and Linear Assignment Problem (LAP) tracker.

### Immunoblotting

The protein expression in cells was determined by western blotting analysis (Ref. 61; incorporated by reference in its entirety). Cells were homogenized in RIPA lysis buffer containing phosphatase/protease cocktail inhibitors (Cell Signaling Technology, #5870). The protein concentration was determined by BCA Protein Assay Kit (Thermo Fisher Scientific, #P123225). Protein solution was mixed with the Leammli sample buffer and heated at 95° C. before loading to SurePAGE gels (GenScript, #M00653). The gels were run at a constant voltage of 80 V and then transferred to 0.2 μm nitrocellulose membrane (Bio-Rad, #1620112) using the Trans-Blot Turbo system (Bio-Rad) with a preprogrammed standard protocol for 30 min. Nitrocellulose membrane was incubated with 5% dry milk to block any unspecific staining. Primary antibodies, including TFPI2 (Abcam, #ab186747), CD133 (Abeam, #ab19898), SOX2 (Abcam, #ab97959), cleaved caspase-3 (Cell Signaling Technology, #9661), P-STAT6 (Abcam, #ab28829), STAT6 (Cell Signaling Technology, #9362), P-STAT3 (Cell Signaling Technology, #9145), STAT3 (Cell Signaling Technology, #9139), P-AKT (Cell Signaling Technology, #4058S), AKT (Bio-Rad, #VMA00253), P-JNK (Cell Signaling Technology, #4668S), JNK (Cell Signaling Technology, #9252S), PLCγ1 (Cell Signaling Technology, #5690S), P-PLCγ1 (Cell Signaling Technology, #8713S), PKCz (Cell Signaling Technology, #9368S), P-PKCz (Cell Signaling Technology, #9378S), Integrin aV/CD51 (Cell Signaling Technology, #4711S), and Actin (Cell Signaling Technology, #3700), were incubated with the membrane overnight. HRP-linked, anti-mouse (Cell Signaling Technology, #7076) or anti-rabbit (Cell Signaling Technology, #7074S) secondary antibodies were applied to the nitrocellulose membrane accordingly. After washing, nitrocellulose membrane was incubated with ECL substrate and imaged under ChemiDoc Touch Imaging System (Bio-Rad).

### Co-Immunoprecipitation (Co-IP)

Co-IP was performed using Invitrogen™ Dynabeads™ Protein G Immunoprecipitation Kit (Thermo Fisher Scientific, #10007D). Briefly, human or mouse microglia were treated with 20 ng/ml TFPI2 recombinant protein for 1 hr. RIPA lysis buffer was then used to extract protein after washing with PBS. Magnetic beads (1.5 mg) were separated from the solution using a magnetic stand. 10 mg TFPI2 antibody (Abcam, #ab186747), CD51 antibody (Cell Signaling Technology, #4711S), or rabbit IgG (isotype control, Cell Signaling Technology, #2729) was prepared in 200 ml of antibody binding and washing Buffer. Antibody or IgG was incubated with magnetic beads for 10 min at room temperature with rotation. After removing the supernatant, antibody/IgG-conjugated magnetic beads were incubated with cell lysis buffer for 2 hrs at room temperature with rotation to immunoprecipitate TFPI2- or CD51-interacting antigens. The magnetic bead-antibody-antigen complex was washed three times using 200 ml washing buffer and then eluted with the elution buffer. The complex was loaded into 4-12% SurePAGE gels to evaluate target protein expression after Co-IP.

### IP-MS Assay

PrhMG were incubated with 20 ng/ml TFPI2 recombinant protein for 1 hr to allow the binding between TFPI2 and its potential receptors. After incubation, cells were pelleted, and membrane-associated proteins were extracted using the Mem-PER™ Plus Membrane Protein Extraction Kit (Thermo Fisher Scientific, #89842). 5×10⁶ PrhMG were resuspended by scraping. After centrifugation at 300 g for 5 min, cells were suspended in cell wash solution and recentrifuged, and then were incubated with 0.75 ml permeabilization buffer for 10 min at 4° C. with constant mixing. Permeabilized cells were centrifuged for 15 min at 16,000 g to remove the supernatant containing cytosolic proteins. The cell pellets were then incubated with 0.5 ml solubilization buffer at 4° C. for 30 min with constant mixing. Solubilized membrane and membrane-associated proteins were isolated by centrifuging at 16,000 g for 15 min at 4° C. Extracted membrane-associated proteins were proceeded for IP-MS pull-down assay. A total of 50 mg cell membrane lysate was incubated with 10 ml TFPI2 antibody (Abcam, #ab186747) or IgG control (Cell Signaling Technology, #2729) for 12 hrs with gentle rotation at 4° C. 100 ml Protein A/G PLUS-Agarose (#sc-2003, Santa Cruz Biotechnology) were mixed with each sample on ice. The lysate bead mixture was incubated at 4° C. under rotary agitation for 4 hrs. The tubes were centrifuged to remove supernatant. The beads were then washed in lysis buffer three times to remove non-specific binding. After removing wash buffer, the complex was washed with pre-urea wash buffer (50 mM Tris pH 8.5, 1 mM EGTA, 75 mM KCl) to remove all residual supernatant. The protein was eluted with the elution buffer (20 mM Tris pH 7.5, and 100 mM NaCl) and sent to the Northwestern Proteomics Core for protein identification. Samples were in-gel digested (trypsin) and subject to mass spectrometry analysis. IP pulled-down samples were analyzed by LC-MS/MS using a Dionex UltiMate 3000 Rapid Separation nanoLC coupled to the Orbitrap Elite Mass Spectrometer (Thermo Fisher Scientific Inc, San Jose, CA). Raw files were searched against a human database and submitted to Mascot and Scaffold searches.

### Calcium Mobilization Assay

Calcium mobilization in microglia was determined using the Fluo-4 Direct™ Calcium Assay Kit (Thermo Fisher Scientific, #F10472). Briefly, microglia with or without CD51 knockdown were seeded into 96-well plates overnight at a concentration of 5000 cells/well with 50 ml complete cell culture media. CD51 inhibitor MK-0429 at distinct concentrations was added into corresponding wells for 1 hr before the testing. 2× Fluo-4 Direct™ calcium reagent loading solution (50 ml) was directly added to wells containing cells in culture media. The plate was incubated at 37° C. for 30 min. 20 ng/ml TFPI2 recombinant protein was added to the plate. Fluorescence kinetic was immediately measured in a microplate reader using instrument settings for excitation at 494 nm and emission at 516 nm.

### Quantitative Real-Time PCR (RT-GPCR)

Cells were pelleted, and RNA was isolated with the RNeasy Mini Kit (Qiagen, #74106). RNA was quantified by NanoDrop spectrophotometers and then reverse-transcribed into cDNA by using the All-In-One 5×RT MasterMix (Applied Biological Materials, #G592) in T00 Thermal Cycler (Bio-Rad). RT-qPCR was performed using SYBR Green PCR Master Mix (Bio-Rad, #1725275) in CFX Connect Real-Time PCR Detection System (Bio-Rad). The expression of each gene was normalized to housekeeping genes. RT-PCR primers are listed in Table 2.

TABLE 2
A list of primers used for RT-qPCR analysis.

| | | |
|---|---|---|
| Mouse Arg1 forward | TTGGGTGGATGCTCACACTG | SEQ ID NO: 1 |
| Mouse Arg1 reverse | GTACACGATGTCTTTGGCAGA | SEQ ID NO: 2 |
| Mouse Cd163 forward | ATGGGTGGACACAGAATGGTT | SEQ ID NO: 3 |
| Mouse Cd163 reverse | CAGGAGCGTTAGTGACAG | SEQ ID NO: 4 |
| Mouse Il10 forward | GCTCTTACTGACTGGCATGAG | SEQ ID NO: 5 |
| Mouse Il10 reverse | CGCAGCTCTAGGAGCATGTG | SEQ ID NO: 6 |
| Mouse Mif forward | GCCAGAGGGGTTTCTGTCG | SEQ ID NO: 7 |
| Mouse Mif reverse | GTTCGTGCCGCTAAAAGTCA | SEQ ID NO: 8 |
| Mouse Il23 forward | AACAACAGCTCGGATTTGGTAT | SEQ ID NO: 9 |
| Mouse Il23 reverse | ATGACCAGGACATTCAGCAGT | SEQ ID NO: 10 |
| Mouse Cxcl11 forward | GGCTTCCTTATGTTCAAACAGGG | SEQ ID NO: 11 |
| Mouse Cxcl11 reverse | GCCGTTACTCGGGTAAATTACA | SEQ ID NO: 12 |
| Human ARG1 forward | TGGACAGACTAGGAATTGGCA | SEQ ID NO: 13 |
| Human ARG1 reverse | CCAGTCCGTCAACATCAAAACT | SEQ ID NO: 14 |
| Human CD163 forward | GCGGGAGAGTGGAAGTGAAAG | SEQ ID NO: 15 |
| Human CD163 reverse | GTTACAAATCACAGAGACCGCT | SEQ ID NO: 16 |
| Human IL10 forward | GACTTTAAGGGTTACCTGGGTTG | SEQ ID NO: 17 |
| Human IL10 reverse | TCACATGCGCCTTGATGTCTG | SEQ ID NO: 18 |
| Human MRC1 forward | TGCTCTACAAGGGATCGGGT | SEQ ID NO: 19 |
| Human MRC1 reverse | ACACGCCAAACAAGAACATGA | SEQ ID NO: 20 |
| Human IL23 forward | AGCAGCAATTAAGAACTGCCA | SEQ ID NO: 21 |
| Human IL23 reverse | TACCAAAGCCGAGCTGTTGTT | SEQ ID NO: 22 |
| Human CXCL11 forward | GACGCTGTCTTTGCATAGGC | SEQ ID NO: 23 |
| Human CXCL11 reverse | GGATTTAGGCATCGTTGTCCTTT | SEQ ID NO: 24 |
| Human IL6 forward | CCTGAACCTTCCAAAGATGGC | SEQ ID NO: 25 |
| Human IL6 reverse | TTCACCAGGCAAGTCTCCTCA | SEQ ID NO: 26 |
| Human TNFA forward | GAGGCCAAGCCCTGGTATG | SEQ ID NO: 27 |
| Human TNFA reverse | CGGGCCGATTGATCTCAGC | SEQ ID NO: 28 |
| Human LFNG forward | GTCAGCGAGAACAAGGTGC | SEQ ID NO: 29 |
| Human LFNG reverse | GATCCGCTCAGCCGTATTCAT | SEQ ID NO: 30 |
| Human HLA-DR forward | TCTGGCGGCTTGAAGAATTTG | SEQ ID NO: 31 |
| Human HLA-DR reverse | GGTGATCGGAGTATAGTTGGAGC | SEQ ID NO: 32 |

### Immunohistochemistry and Immunofluorescence

Immunohistochemistry and immunofluorescence were performed using a standard protocol (Ref. 61; incorporated by reference in its entirety). Paraffin sections were baked at 65° C. for 2 hrs prior to staining. Sections were then subjected to xylene and ethanol in gradient concentrations for deparaffinization and rehydration. Permeabilization buffer containing 0.3% (v/v) Triton X-100 and 1% (v/v) H₂O₂ was added for 30 min to block endogenous peroxidase. Antigen retrieval was conducted by boiling sections in the sodium citrate buffer (0.01 M, pH=6). After washing with PBS, tissues were blocked by 5% goat serum for 30 min. Sections were incubated with primary antibody for 1 hr at room temperature and then overnight at 4° C. Sections were subjected to corresponding secondary antibodies or rabbit-on-rodent HRP-polymer (Biocare, #RMR 622) for 1 hr at room temperature. For immunofluorescence, cell nucleus was counterstained with DAPI/anti-fade mounting medium (Vector Laboratories, #H-1200-10). Protein signal was captured using Nikon AX/AX R Confocal Microscope System in the Center for Advanced Microscopy (CAM) at Northwestern University. The relative intensity of protein was determined by Image J. For immunohistochemistry, tissues were incubated with DAB Quanto (Epredia, #TA125QHDX). The nucleus was then stained by hematoxylin. After dehydration, the images were captured by using EVOS Cell Imaging System. ImageJ with IHC profiler plug-in was used for scoring positive signals (Ref. 77; incorporated by reference in its entirety).

### Hematoxylin and Eosin (H&E) Staining

H&E staining was performed using the staining kit (Abcam, #ab245880) Paraffin sections were baked at 65° C. for 2 hrs prior to staining. Sections were then subjected to xylene and ethanol in gradient concentrations for deparaffinization and rehydration. After washing two times in distilled water, sections were incubated with hematoxylin, Mayer's (Lillie's Modification) for 5 min. Slides were rinsed in distilled water to remove excess stains. Sections were incubated with the Bluing Reagent for 15 sec, and then the Eosin Y Solution (Modified Alcoholic) for 3 min. After washing, slides were dehydrated in three changes of absolute alcohol. The images of tissue sections were captured by TissueFAXS in the Center for Advanced Microscopy (CAM) at Northwestern University.

### Human Phospho-Kinase Array

After starvation for 24 hrs followed by TFPI2 recombinant protein treatment for 1 hr, HMC3 cell lysate was prepared using a lysis buffer with 10 mg/ml aprotinin, 10 mg/ml leupeptin, and 10 mg/ml pepstatin. Phosphorylation profiles of kinases were determined using Proteome Profiler Human Phospho-Kinase Array Kit (Bio-Techne, #ARY003C) as the instruction manual. Signal was detected under ChemiDoc Touch Imaging System (Bio-Rad).

### Brain Tumor and Spleen Cell Isolation

At the endpoint of experiments, mice were sacrificed to harvest their brains and spleens. Immune cells in the brain and spleen were isolated (Ref. 11; incorporated by reference in its entirety). After perfusion with PBS, brains and spleens were homogenized in 10 ml HBSS or 2% FBS RPMI 1640 media on ice, respectively. Brain/spleen-derived immune cells were then purified using 30/70 Percoll (GE Healthcare, #17-0891-01) with gradient separation for 30 min at 4° C. Spleen cells were treated with 1 ml ACK buffer (Thermo Fisher, #A1049201) for 5 min to remove red blood cells. After deactivation in complete RPMI 1640 media, cells were filtered through 70 μm strainers (Thermo Fisher, #08-771-2) and ready for further analysis.

### T Cell-Mediated Cytotoxicity Assay

T cell-mediated cytotoxicity was determined by Cytotox96 non-radioactive cytotoxicity assay kit (Promega, #G1780) based on the release of lactate dehydrogenase (LDH). After expansion and activation, mouse primary CD8⁺ T cells or human JURKAT T cells were collected. About 10⁵ T cells were co-cultured with SIM-A9 microglia or PrhMG (1:1 ratio) treated with TFPI2 recombinant protein in the presence or absence of STAT6 inhibitor AS1517499 for 24 hrs. T cells (effector cells) were then collected for further co-culturing with CT2A cells or GSC272 cells (target cells) at different ratios (10000:1000, 5000:1000, 2500:1000, 1250:1000, 620:1000, 310:1000, 160:1000, 80:1000, 40:1000, 20:1000) for 5 hrs. Experimental groups and corresponding control groups, including effector cell spontaneous LDH release, target cell spontaneous LDH release, target cell maximum LDH release, volume correction control, and culture medium background control were set up following the manufacturer's instructions to evaluate T cell-mediated cytotoxicity on GSCs. Optical density (OD) was measured by a Biotek Synergy 2 SL Microplate Reader. The cytotoxicity was calculated by the formula: (experimental OD value-effector spontaneous OD value-target spontaneous OD value)/(target maximum OD value-target spontaneous OD value).

### Apoptosis Analysis

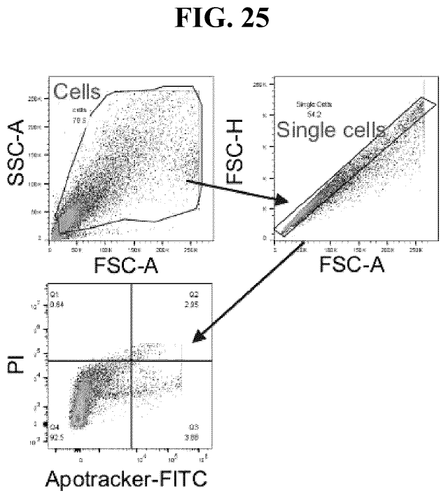

Cell apoptosis was evaluated using Apotracker Green (BioLegend, #427402) (Ref. 78; incorporated by reference in its entirety). 10⁵ cells were collected and stained with Apotracker (1:10 dilution) in 100 ml cell staining buffer. 5 ml propidium iodide (PI) solution (BioLegend, #421301) was added for labeling late apoptotic and necrotic cells. After washing with PBS twice, FITC and PI signals were analyzed in BD FACSymphony flow cytometer. The gating strategy for apoptosis analysis is shown in FIG. 25.

### Flow Cytometry

For membrane protein staining, the single-cell suspensions were washed with PBS and then incubated with fixable viability dye (Invitrogen, #5211229035) on ice for 10 min. Following washing with PBS, cells were incubated with the anti-CD16/CD32 cocktail (BioLegend, #103132) in 2% BSA PBS to block Fc receptors for 30 min on ice. Different antibody combinations, including Percp/Cy5.5 anti-mouse CD45 (BioLegend, #103132), AF488 anti-mouse CD3 (BioLegend, #100210), BV421 anti-mouse CX3CR1 (BD Bioscience, #567531), BUV395 anti-mouse CD4 (BD Bioscience, #740208), BV711 anti-mouse CD8 (BioLegend, #100747), PE/Cy7 anti-mouse/human CD11b (BioLegend, #101216), PE anti-mouse CD68 (BD Bioscience, #566386), PE anti-mouse PD1 (BioLegend, #135205), AF647 anti-mouse CD206 (BD Bioscience, #565250), AF647 anti-human CD206 (BioLegend, #321116), PE anti-human CD69 (BioLegend, #310906), PE/Cy7 anti-mouse CD69 (BioLegend, #104512), and BV650 anti-human HLA-DR (BioLegend, #307650) were added to cell suspensions for 30 min on ice. CD51 (Cell Signaling Technology, #4711S) was added to cell suspension with corresponding antibody combinations for 30 min on ice, followed by incubation of goat anti-rabbit IgG cross-adsorbed secondary antibody (AF594) for 30 min. After washing in PBS, cells were fixed in fixation buffer (BioLegend, #420801) overnight. Samples were read in BD FACSymphony flow cytometer and analyzed in FlowJo v10.8.1.

Intracellular protein staining was performed separately or followed by cell surface marker staining. Cells were fixed in fixation buffer at room temperature for 20 min. After washing in permeabilization buffer (0.1% Triton X-100 in PBS) for two times, cells were incubated with AF700 anti-human IFN-7 (BioLegend, #502520) or FITC anti-human/mouse ARG1 (BioLegend, #IC5868F) for 20 min at room temperature. Then cell suspensions were rewashed in permeabilization buffer and read in BD FACSymphony flow cytometer.

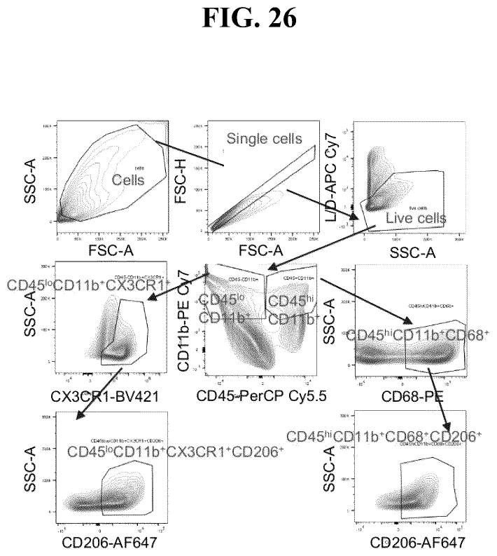

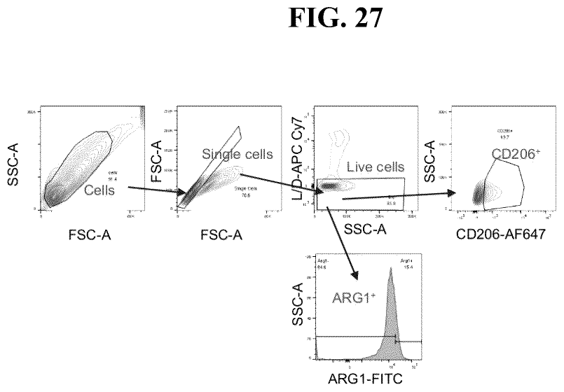

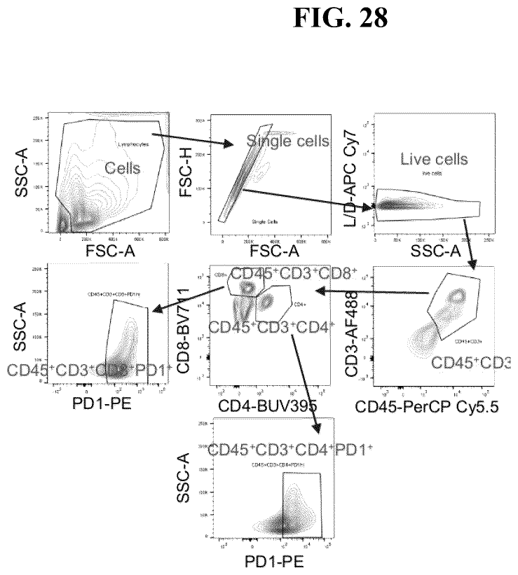

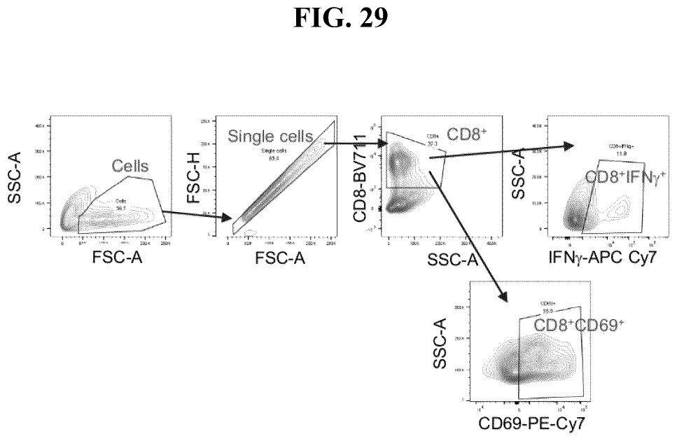

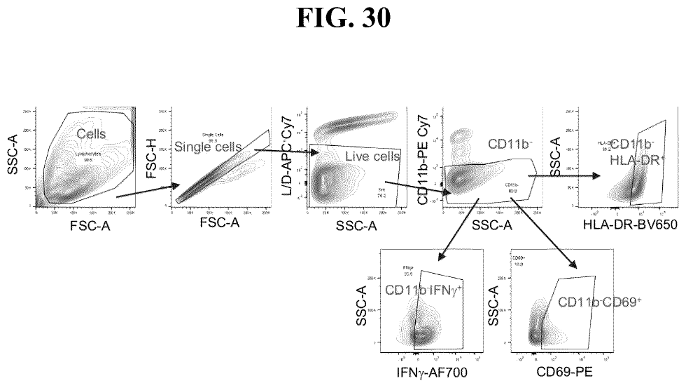

Phospho-flow samples were fixed in a fixation buffer at room temperature for 15 min. Cells were then stained with a fixable viability dye and permeabilized in 90% cold methanol for 15 min on ice. After blocking Fc receptors, cells were resuspended with P-STAT6 primary antibody for 1 hr on ice. Cells were washed in PBS three times and incubated with goat anti-rabbit IgG cross-adsorbed secondary antibody (AF594). Cells were centrifuged at 300 g for 5 min to remove supernatant and washed in PBS twice before the flow cytometry analysis. The gating strategies for flow cytometry analyses are shown in FIGS. 26-30.

### Proliferation Analysis

Cell proliferation was assessed using the CellTrace CFSE Cell Proliferation Kit (Invitrogen, #C34554). 10⁶ cells were harvested and incubated with CFSE working solution (1:1000) for 20 min at 37° C. The staining was stopped by adding complete cell culture media. After washing, cells were continuously cultured for 5 days in the dark. At the end of the incubation, cells were collected by spinning down at 1500 rpm for 5 min. The cell pellet was resuspended in cell staining buffer for flow cytometry analysis. The percentage of CFSE positive peaks over the undivided peak (generation 0) was analyzed using the ImageJ software.

### Tumorsphere Formation Assay

The tumorsphere formation assay was performed (Ref. 79; incorporated by reference in its entirety). GSCs were treated with different concentrations of recombinant TFPI2 protein for 24 hrs with or without inhibition of JNK or STAT3, or modified with TFPI21 KO, knockdown or overexpression. After treatment or genetic modifications, cells were centrifuged at 1500 rpm for 5 min and resuspended in the plain cell culture media without additional supplements. Cells were then seeded into a 96-wells plate at 100 cells/well. The number of tumorspheres in each well was imaged and quantified after 10 days.

### Enzyme-Linked Immunoassay

The levels of TFPI2 in human plasma were measured by enzyme-linked immunoassay (ELISA) using the commercial TFPI2 kit (MyBioSource, #MBS2507217) following the manufacturer's instructions. OD value was measured by a Biotek Synergy 2 SL Microplate Reader.

Bulk RNA-Seq and scRNA-Seq Data Analysis

Extracted RNA from shC and shTFPI2 GSC272 cells were processed to Oligo-dT based library and analyzed using a NovaSeq 6000 instrument to generate RNA-seq dataset at The University of Chicago Functional Genomics (RRID:SCR_019196). Raw FASTQ data from two flowcells per sample was uploaded to Galaxy server (Ref. 80; incorporated by reference in its entirety). Trimmomatic was performed to read and trim paired-end reads. Read quality reports were generated with FastQC tool. HISAT2 program was used to align the data where hg38 was selected as the reference genome. Samtools merge was performed on BAM files to merge data from different flowcells. Gene expression was then determined by FeatureCounts. AnnotateMyIDs program was executed to generate the annotation file. Limma was used to calculate differential expression genes and generate DE table. The up-regulated and downregulated pathways were determined by GSEA analysis. scRNA-seq data of GSE131928 (Ref. 30; incorporated by reference in its entirety) was used for analyzing the expression of TFPI2 and stemness signature in malignant cells, and the scRNA-seq data of EGAS00001004422 (Ref. 81; incorporated by reference in its entirety) and GSE131928 (Ref. 30; incorporated by reference in its entirety) was used for analyzing microglia signature in GBM patient tumors with TFPI2 high versus low in GBM cells.

### Computational Analysis of Human GBM Datasets

For bioinformatics analyses of human GBM patient tumor data, the microarray gene expression and survival data of TCGA dataset or other available datasets was downloaded from GlioVis: gliovis.bioinfo.cnio.es/. The analyses of gene expression, signature expression, correlation, survival, and the GSEA of interesting gene signatures in IDH-WT GBM patients were performed (Refs. 11-12, 61; incorporated by reference in their entireties).

### Mice and Intracranial Xenograft Tumor Model

Female C57BL/6 (#0000664), nude mice (#007850), and B6.12952(C)-Stat6tm1Gru/J mice (STAT6-KO; #005977) at 6 weeks of age were purchased from the Jackson Laboratory. Mice were grouped by 5 animals and maintained in a pathogen-free IVC System (San Diego, CA) for a week before the experiment. All animal experiments were performed with the approval of the Institutional Animal Care and Use Committee (IACUC) at Northwestern University. The intracranial xenograft tumor models were established (Refs. 11, 61; incorporated by reference in their entireties). Ketamine (100 mg/kg) and xylazine (20 mg/kg) were used for anesthetizing mice through intraperitoneal injection. Then a dental drill was used to open a small hole in the skull 1.2 mm anterior and 3.0 mm lateral to the bregma. A total volume of 5 mL cells in FBS-free DMEM was injected into the right caudate nucleus 3 mm below the surface of the brain using a 10 mL Hamilton syringe with an unbeveled 30-gauge needle. Total three doses of 20 mg/kg meloxicam were administrated before and after surgery through subcutaneous injection. After cancer cell implantation, mice were monitored for recording survival. Mice with neurological deficits or moribund appearance were sacrificed. Following the transcardiac perfusion with PBS and 4% paraformaldehyde (PFA), brains were removed and fixed in 4% PFA, and were processed for paraffin-embedded blocks or cryosectioning.

### Patient Samples

GBM patient peripheral blood plasma samples (n=55) were collected from the Northwestern Central Nervous System Tissue Bank (NSTB). Patient tumor samples (n=60) from surgically-resected IDH-WT GBMs were collected at the NSTB. All patients were diagnosed according to the WHO diagnostic criteria by neuropathologist Dr. Craig Horbinski. Detailed patient information is provided in Table 3. Control plasma samples (n=6) from healthy human blood were purchased from Solomonpark (#4345), which are commercially available anonymized and de-identified. According to The George Washington University Institutional Review Board and based on the guidelines from the Office of Human Research Protection, the conducted research meets the criteria for exemption #4 (45 CFR 46.101(b) Categories of Exempt Human Subjects Research) and does not constitute human research.

TABLE 3
Patient information.

| | | | New | | | | |
|---|---|---|---|---|---|---|---|
| | | | diagnosis | | | | |
| | Age at | | vs. | Overall | | | |
| | time of | | recurrent | survival - | Ki-67 | | IHC |
| NSTB ID | collection | Sex | diagnosis | days | Histology | ELISA | staining |
| GBM | | | | | | GBM | GBM |
| NU00538 | 49 | Male | New | 422 | 30% | NU00323 | NU00538 |
| NU01713 | 51 | Male | New | 1025 | 30% | NU00538 | NU00696 |
| NU01793 | 66 | Female | New | | 40% | NU00655 | NU01063 |
| NU01798 | 70 | Female | New | | 30% | NU00677 | NU01094 |
| NU01808 | 37 | Female | New | 470 | 40-50% | NU00761 | NU01115 |
| NU01853 | 66 | Female | New | 370 | Not reported | NU00764 | NU01126 |
| NU01861 | 55 | Male | New | 297 | 60% | NU00792 | NU01143 |
| NU01903 | 62 | Male | New | 434 | 60-70% | NU00908 | NU01161 |
| NU01991 | 62 | Male | New | 138 | >80% | NU00915 | NU01197 |
| NU01994 | 80 | Male | New | | 8-10% | NU01063 | NU01220 |
| NU02013 | 66 | Male | New | 389 | 20% | NU01107 | NU01249 |
| NU02064 | 62 | Female | New | | 0.5 | NU01135 | NU01260 |
| NU02120 | 69 | Female | New | | 0.2 | NU01185 | NU01322 |
| NU02156 | 63 | Male | New | | 0.4 | NU01197 | NU01324 |
| NU02203 | 76 | Female | New | 233 | 0.25 | NU01201 | NU01327 |
| NU02411 | 59 | Female | New | 389 | 0.4 | NU01226 | NU01352 |
| NU02446 | 58 | Female | New | | 0.4 | NU01232 | NU01363 |
| NU02541 | 68 | Female | New | | 0.15 | NU01247 | NU01482 |
| NU02569 | 70 | Male | New | | 0.25 | NU01276 | NU01485 |
| NU02647 | 67 | Male | New | | 0.3 | NU01293 | NU01520 |
| NU01063 | 69 | Female | Recurrent | 339 | 20% | NU01327 | NU01571 |
| NU01197 | 64 | Female | Recurrent | 874 | 0.1 | NU01485 | NU01584 |
| NU01327 | 63 | Male | Recurrent | 805 | Not reported | NU01620 | NU01631 |
| NU01485 | 24 | Female | Recurrent | | 80-90% | NU01702 | NU01644 |
| NU02209 | 58 | Female | Recurrent | 675 | 0.7 | NU01713 | NU01670 |
| NU02254 | 60 | Female | Recurrent | 746 | 0.5 | NU01743 | NU01713 |
| NU02326 | 64 | Male | Recurrent | | 0.6 | NU01761 | NU01793 |
| NU02359 | 76 | Female | Recurrent | 149 | 0.4 | NU01793 | NU01798 |
| NU02576 | 55 | Female | Recurrent | | 30% | NU01798 | NU01808 |
| NU02718 | 60 | Male | Recurrent | | 30-40% | NU01808 | NU01853 |
| NU00323 | 46 | Male | Recurrent | 1086 | 5% | NU01853 | NU01861 |
| NU00677 | 72 | Female | New | 311 | 20% | NU01861 | NU01903 |
| NU00792 | 77 | Male | New | 54 | 20% | NU01903 | NU01918 |
| NU00908 | 39 | Male | New | 1094 | 20% | NU01967 | NU01928 |
| NU01107 | 62 | Female | New | 401 | 15% | NU01991 | NU01991 |
| NU01185 | 73 | Male | Recurrent | | 1-2% | NU01994 | NU01994 |
| NU01201 | 83 | Female | New | 434 | Not reported | NU02013 | NU02013 |
| NU01226 | 83 | Male | New | 189 | 20% | NU02064 | NU02064 |
| NU01232 | 76 | Male | New | 231 | 35% | NU02120 | NU02068 |
| NU01247 | 51 | Male | New | 691 | 40% | NU02156 | NU02120 |
| NU01276 | 45 | Male | Recurrent | 673 | Not reported | NU02203 | NU02156 |
| NU01293 | 68 | Female | Recurrent | 1050 | 30-40% | NU02209 | NU02203 |
| NU01620 | 71 | Male | New | 381 | 20% | NU02254 | NU02209 |
| NU01702 | 53 | Male | Recurrent | 386 | 2% | NU02299 | NU02254 |
| NU01743 | 74 | Male | New | 102 | 30% | NU02326 | NU02267 |
| NU01761 | 66 | Male | Recurrent | | 30% | NU02337 | NU02326 |
| NU01967 | 84 | Female | New | 379 | 50% | NU02359 | NU02359 |
| NU02299 | 56 | Male | New | | 30% | NU02411 | NU02411 |
| NU02337 | 50 | Male | New | | 50% | NU02446 | NU02446 |
| NU02618 | 76 | Male | New | | 25% | NU02541 | NU02576 |
| NU00655 | 53 | Female | New | 959 | 10% | NU02569 | NU02594 |
| NU00761 | 49 | Male | New | 378 | 20% | NU02576 | NU02674 |
| NU00764 | 78 | Female | New | 283 | 15% | NU02618 | NU02684 |
| NU00915 | 79 | Female | New | 31 | 50% | NU02647 | NU02718 |
| NU01135 | 48 | Female | Recurrent | 289 | 5% | NU02718 | NU02829 |
| NU00696 | 64 | Male | New | 122 | 20% | | NU03043 |
| NU01094 | 56 | Male | New | 825 | 40% | | NU03053 |
| NU01115 | 64 | Female | New | 998 | 20% | | NU03061 |
| NU01126 | 63 | Male | New | 561 | 30% | | NU03349 |
| NU01143 | 69 | Female | New | 571 | 40% | | NU03352 |
| NU01161 | 74 | Female | New | 622 | 40% | | |
| NU01220 | 60 | Male | Recurrent | 819 | 25% | | |
| NU01249 | 77 | Female | New | 722 | 40% | | |
| NU01260 | 63 | Male | Recurrent | 250 | 10% | | |
| NU01322 | 67 | Female | New | 604 | 20% | | |
| NU01324 | 74 | Female | New | 285 | Not reported | | |
| NU01352 | 68 | Female | New | 548 | 20% | | |
| NU01363 | 56 | Male | New | 589 | 40% | | |
| NU01482 | 26 | Female | New | 999 | 80% | | |
| NU01520 | 62 | Male | New | | 40% | | |
| NU01571 | 72 | Male | New | 273 | Not reported | | |
| NU01584 | 80 | Male | New | 15 | 40% | | |
| NU01631 | 52 | Male | New | | 40% | | |
| NU01644 | 66 | Female | New | | 15% | | |
| NU01670 | 63 | Female | New | 284 | Not reported | | |
| NU01918 | 61 | Male | New | 474 | 25% | | |
| NU01928 | 80 | Female | New | 202 | 40% | | |
| NU02068 | 65 | Male | Recurrent | 874 | 70% | | |
| NU02267 | 51 | Female | New | 460 | 20% | | |
| NU02594 | 90-95 | Male | New | 20 | 80% | | |
| NU02674 | 54 | Female | New | | 25% | | |
| NU02684 | 49 | Male | Recurrent | 824 | not reported | | |
| NU02829 | 65 | Female | New | | not reported | | |
| NU03043 | 63 | Female | Recurrent | | not reported | | |
| NU03053 | 74 | Female | Recurrent | 644 | not reported | | |
| NU03061 | 52 | Male | New | | 10-15% | | |
| NU03349 | 61 | Male | New | 66 | 50-60% | | |
| NU03352 | 36 | Female | New | 61 | 30% | | |

### Statistical Analysis

Statistical analysis was conducted using GraphPad Prism 9 (GraphPad Software, USA). In vitro and in vivo measurement data were presented as the means±SEM or SD. Comparison between two groups was evaluated using Student's t-test, and multiple comparisons among groups were evaluated using a one-way ANOVA test in Tukey's method. The survival analysis for animal models was performed using Log-rank (Mantel-Cox) test. Correlation analysis was performed using the Pearson test to determine the R and P values. Differences with a minimum of P<0.05 were indicated as statistically significant.

### Results

### TFPI2 Overexpression Promotes GSC Self-Renewal.

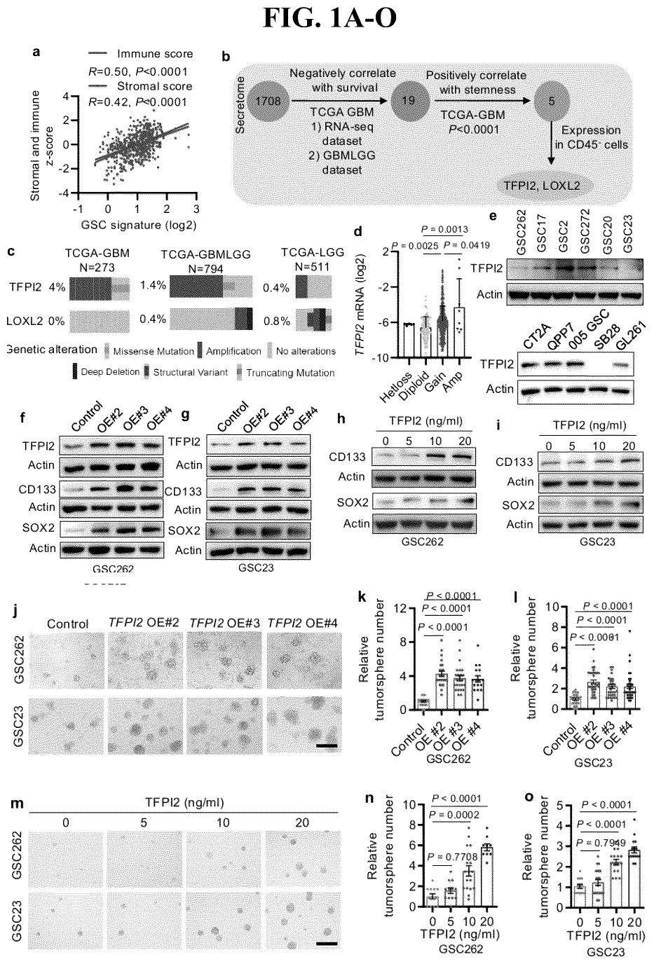

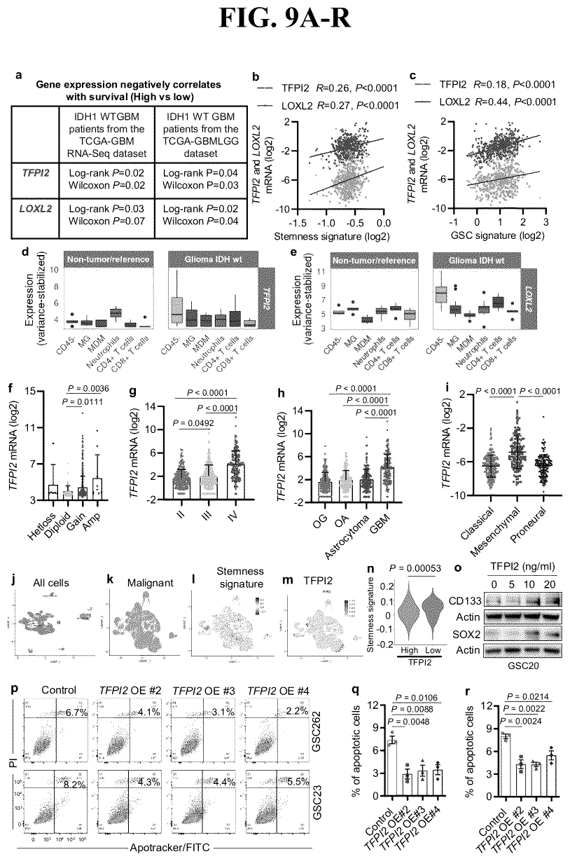

Pan-cancer analyses have shown that high stemness of cancer cells is correlated positively with increased immunosuppressive pathways (Ref. 25; incorporated by reference in its entirety). To identify potential associations between GSCs and immunity, correlation analyses were performed between tumor stromal and immune cell scores (Ref. 26; incorporated by reference in its entirety) and GSC signature (Ref. 27; incorporated by reference in its entirety) using The Cancer Genome Atlas (TCGA) GBM dataset. It was found that GSC signature correlated positively with both stromal and immune scores (FIG. 1a). To identify the key gene/factor(s) that could mediate the GSC-immune cell crosstalk, the following approaches were used: 1) start with the 1,708 human genes that encode secreted proteins (Ref. 28; incorporated by reference in its entirety); 2) determine the subset of these genes whose expression inversely correlates with TCGA GBM patient survival; 3) determine the subset of these genes whose expression directly correlates with tumor stemness signature (Ref. 25; incorporated by reference in its entirety); and 4) rank the expression of these genes in CD45⁻ GBM cells/GSCs using the Brain Tumor Immune Micro Environment (TIME) dataset (Ref. 29; incorporated by reference in its entirety). Following these analyses, TFPI2 and LOXL2 were identified (FIG. 1b and FIG. 9a-e). Analysis of TCGA GBM data for additional information about these two genes revealed that TFPI2, but not LOXL2, is amplified in approximately 4% of GBM cases, 1.4% of GBM and low-grade glioma (LGG) merged cases, and 0.4% of LGG cases (FIG. 1c), and the enhanced TFPI2 gene copy number correlated positively with increased TFPI2 mRNA level in GBM (FIG. 1d and FIG. 9f). In addition, the expression of TFPI2 was found to be higher in GBM than LGG (FIG. 1g,h). With respect to a commonly used transcriptional subclassification of GBM, mesenchymal GBM showed the highest average level of TFPI2 expression (FIG. 9i).

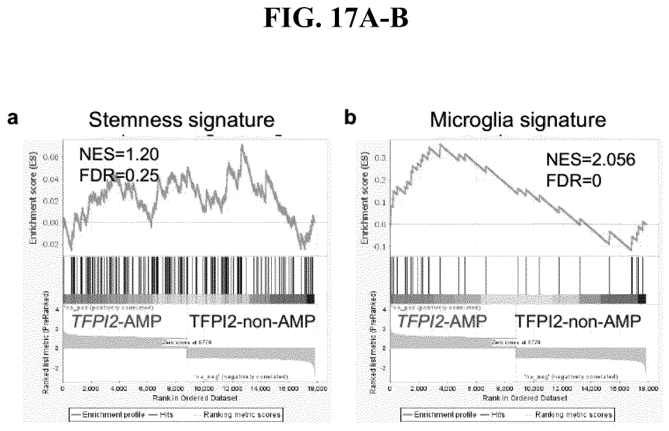

To investigate a possible relationship between TFPI2 expression and GSC self-renewal, TCGA GBM data was analyzed by comparing TFPI2-amplified patient tumors, that have elevated TFPI2 expression, versus non-amplified tumors for stemness signature. The result of this analysis showed that the stemness signature was enriched in TFPI2-amplified tumors (FIG. 17a). Single-cell RNA sequencing (scRNA-seq) data was next analyzed from 28 patient GBM (Ref. 30; incorporated by reference in its entirety) and found that the stemness signature was enriched in TFPI2-high GBM cells (FIG. 9j-n). To address the role of TFPI2 in regulating GSC stemness through experimentation, TFPI2 was overexpressed or TFPI2 recombinant protein was added to patient-derived GSCs with relatively low TFPI2 expression, such as GSC262, GSC23, and GSC20 (FIG. 1e). Western blotting showed that ectopic TFPI2 expression (FIG. 1f,g) and TFPI2 recombinant protein treatment (FIG. 1h,i and FIG. 9o) increased the expression of stemness-associated factors CD133 and SOX2. The ectopic expression of TFPI2 increased GSC self-renewal (FIG. 1j-1) while decreasing GSC apoptosis (FIG. 9p-r). Enhanced self-renewal was also observed in GSCs upon TFPI2 recombinant protein treatments (FIG. 1m-o). Together, these findings demonstrate that TFPI2 is amplified in GBM, and its overexpression promotes GSC self-renewal.

### Genetic Depletion of TFPI2 Impairs GSC Self-Renewal and Extends Survival of GSC-Bearing Mice

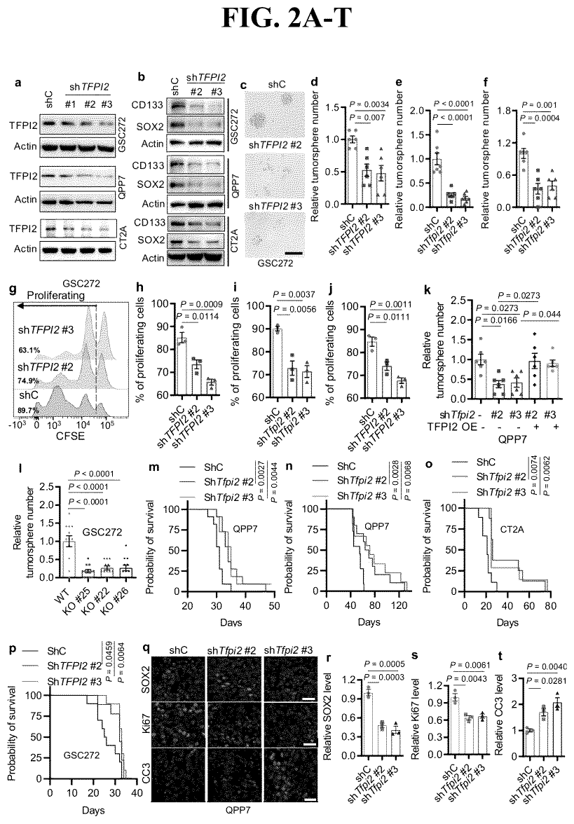

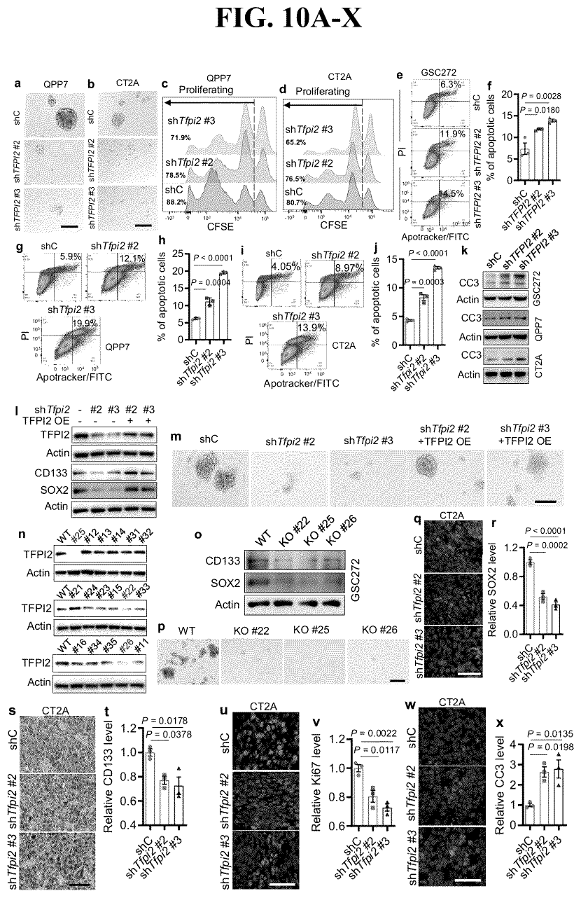

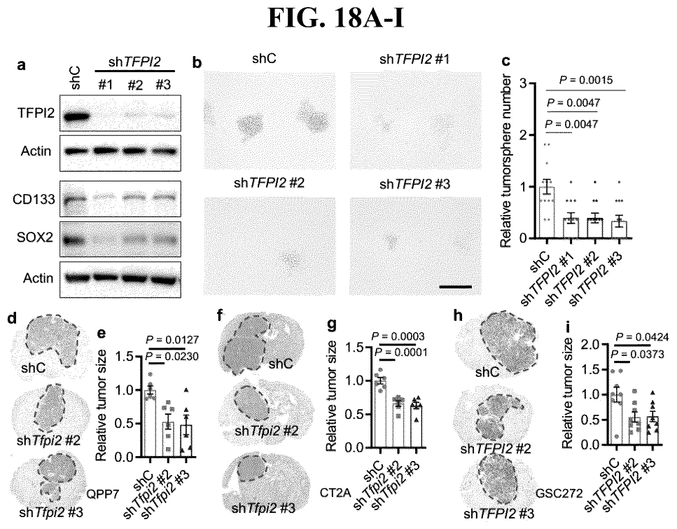

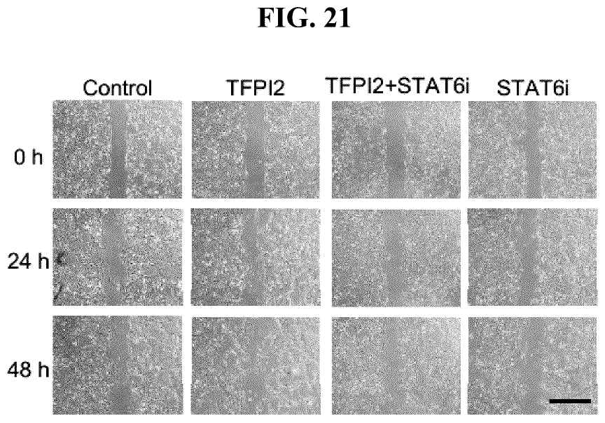

To address the effects of reducing cellular TFPI2 in GSC sternness maintenance, TFPI2 was targeted by shRNA-mediated knockdown (FIG. 2a and FIG. 18a) in human and mouse GSCs with relatively high endogenous TFPI2 expression (FIG. 1e), such as GSC272 and GSC2 (GBM patient-derived GSCs), QPP7 GSCs (a mouse GSC line derived from an engineered GBM mouse model with null alleles for Qki, Pten, and Trp53), and CT2A cells (a mouse line isolated from a carcinogen-induced glioma possessing a GSC-like phenotype (Refs. 11, 32; incorporated by reference in their entireties)). shRNA-mediated depletion of TFPI2 resulted in reduced CD133 and SOX2 expression (FIG. 2b and FIG. 18a), self-renewal (FIG. 2c-f, FIG. 10a,b and FIG. 18b,c) and proliferation (FIG. 2g-j and Data FIG. 10c,d) in both human and mouse GSCs. Conversely, the cellular apoptosis was increased upon TFPI2 knockdown in GSCs (10 FIG. 2e-k). Reexpression of shRNA-resistant TFPI2 cDNA rescued TFPI2 depletion-induced impairment of CD133 and SOX2 expression as well as self-renewal (FIG. 2k and FIG. 10l,m). Lastly, the role of TFPI2 in promoting GSC stemness was reinforced by an additional genetic approach showing that CRISPR-mediated TFPI2 knockout (KO) in GSC272 reduced CD133 and SOX2 expression as well as self-renewal (FIG. 21 and FIG. 10n-p).

To further investigate the role of TFPI2 in GBM tumor in vivo, the shRNA knockdown system was utilized to deplete TFPI2 in QPP7 and CT2A tumors implanted into C57BL/6 mice and in GSC272 tumors implanted into nude mice. TFPI2 depletion significantly inhibited tumor growth (FIG. 18d-i) and extended the survival of tumor-bearing mice (FIG. 2m-p). Immunohistochemical analysis of tumors revealed that GSC markers SOX2 and CD133 (FIG. 2q,r and FIG. 10q-t) and proliferation marker Ki67 (FIG. 2q,s and FIG. 10u,v) were significantly reduced, whereas apoptosis marker cleaved caspase 3 (FIG. 2q,t and FIG. 10w,x) was increased, in tumors established from TFPI2-depleted GSCs. Collectively, these results confirm the role of TFPI2 in promoting GBM tumor progression, which correlates with TFPI2-induced enhancement of GSC stemness, proliferation, and survival.

### TFPI2 Activates JNK-STAT3 Signaling to Promote GSC Self-Renewal and Tumor Growth.

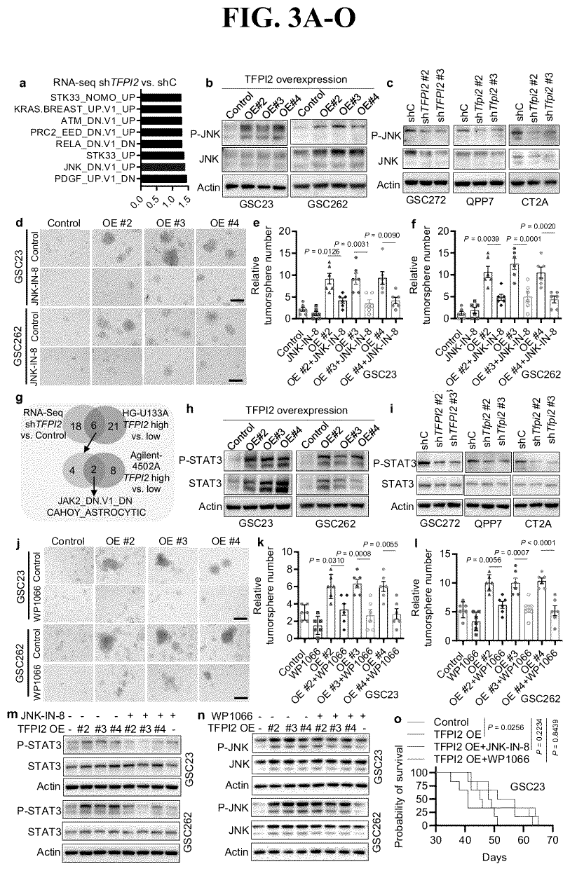

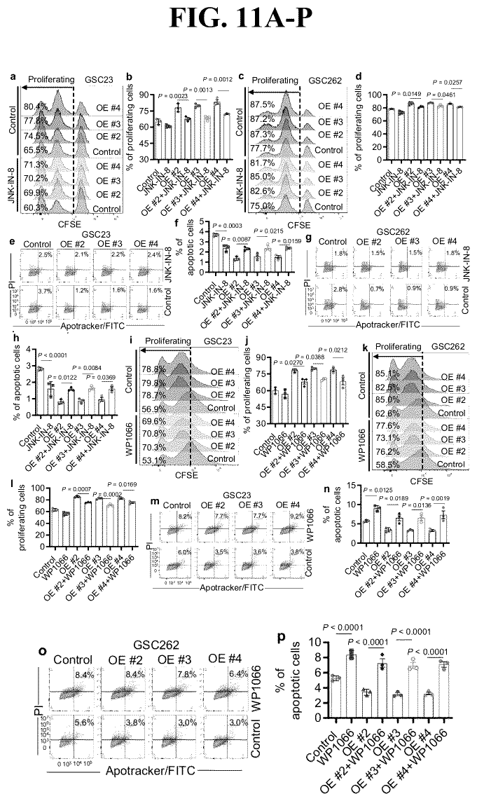

To determine the molecular basis of TFPI2's effect on GSC self-renewal, RNA-seq profiling was performed on GSC272 cells with or without TFPI2 knockdown. Gene Set Enrichment Analysis (GSEA) on oncogenic signatures resulted in identification of 8 signatures that were influenced by TFPI2 (FIG. 3a). Among them, JNK (one of the top two pathways), that has been associated with tumor cell sternness (Refs. 33-34; incorporated by reference in its entirety) was examined by immunoblotting with results showing that increasing GSC TFPI2 expression promoted corresponding increases in JNK and P-JNK; and the opposite effect was apparent in TFPI2 knockdown GSCs (FIG. 3b,c). Pharmacologic inhibition of JNK with its inhibitor JNK-IN-8 negated self-renewal (FIG. 3d-f), proliferation (FIG. 11a-d), and apoptotic (FIG. 11e-h) effects of increasing cellular TFPI2 in GSCs.

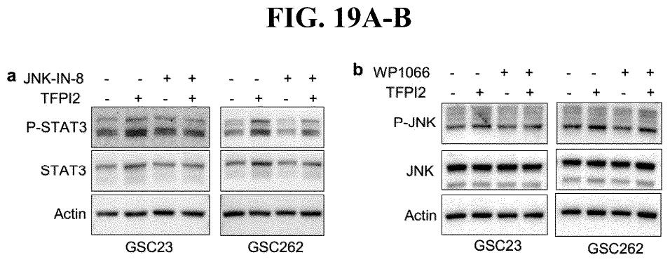

TCGA GBM datasets (HG-U133A and Agilent-4502A) with TFPI2-high patient tumors versus TFPI2-low patient tumors and GSC272 RNA-Seq data with shTFPI2 versus shRNA control (shC) were analyzed. GSEA on oncogenic signatures resulted in identification of two key signatures that were influenced by TFPI2 expression (FIG. 3g). One of these, JAK2/STAT3, has been described as being essential for GSC self-renewal (Refs. 35-36; incorporated by reference in its entirety). Consistent with the results from the JNK pathway analysis, elevated STAT3 and P-STAT3 were observed in association with increased TFPI2 expression in GSCs, whereas TFPI2 depletion had an opposite effect (FIG. 3h,i). Treatment of GSCs with STAT3 inhibitor WP1066 negated the self-renewal (FIG. 3j-1), proliferation (FIG. 11i-1), and apoptotic (FIG. 11m-p) effects of elevating cellular TFPI2. To reveal the connection between JNK and STAT3, TFPI2-overexpressing or TFPI2 recombinant protein-conditioned GSCs were treated with or without JNK-IN-8 or WP1066. Immunoblot analysis revealed that TFPI2-induced STAT3 activation was blocked by JNK inhibition (FIG. 3m and FIG. 19a). However, STAT3 inhibition had no effect on TFPI2-induced JNK activation (FIG. 3n and FIG. 19b). In vivo, TFPI2 overexpression shortened the survival of GSC23 tumor-bearing mice, an effect that was abolished by the treatment with JNK-IN-8 or WP1066 (FIG. 3o). These findings indicate that the JNK-STAT3 signaling pathway is essential for TFPI2-induced GSC self-renewal and tumor growth.

### TFPI2 Promotes Microglia Infiltration Via Activation of STAT6 Signaling.

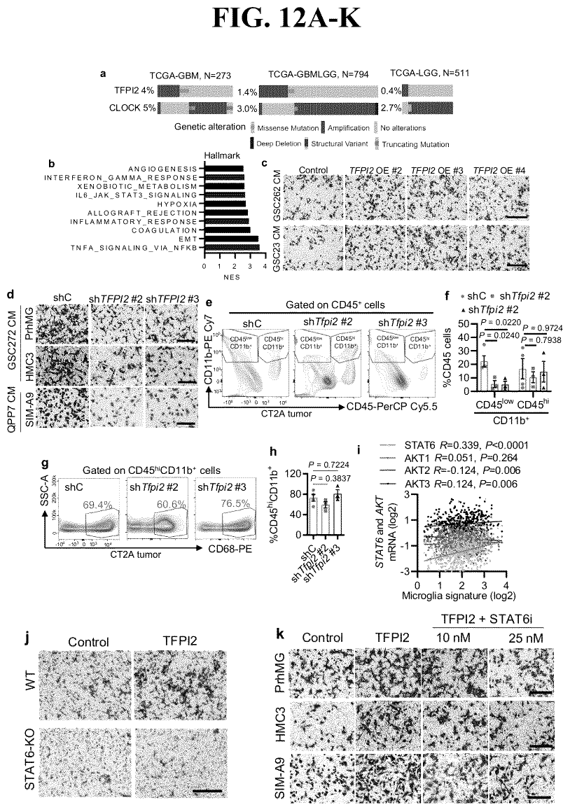

Emerging evidence demonstrates that cancer cell stemness is directly correlated with immunosuppression across cancer types, including GBM (Refs. 7, 25; incorporated by reference in its entirety). CLOCK is a key transcription factor that is amplified in about 5% of GBM cases and acts as a positive regulator of GSC stemness and microglia infiltration (Ref. 11-12; incorporated by reference in their entireties). Analysis of TCGA GBM and LGG datasets demonstrated that TFPI2 amplification is mutually exclusive with CLOCK amplification (FIG. 12a), which may indicate that TFPI2 and CLOCK share similar functions and/or are associated with a common signaling pathway. GSEA on hallmark, GO Biological Process, GO Molecular Function, and KEGG pathways revealed a prominent representation of immune suppressive signatures, cytokine and chemokine signatures, and immune response and leucocyte migration signatures in TFPI2-high GBM patient tumors (FIG. 12b and Table 1). Moreover, microglia signature was enriched in TFPI2-amplified patient tumors compared to control tumors (FIG. 17b).

TABLE 1
Computational analysis demonstrates a key role of TFPI2
for myeloid/leukocyte migration in TCGA GBM patients.

| Top ten | | | |
|---|---|---|---|
| enriched | Gene Ontology | Gene Ontology | |
| pathways | Biological Process | Molecular Function | KEGG Enrichment Analysis |
| #1 | GRANULOCYTE_{—} | SERINE_TYPE_{—} | CYTOKINE_RECEPTOR_{—} |
| | MIGRATION | ENDOPEPTIDASE | INTERACTION |
| #2 | ADAPTIVE_IMMUNE_{—} | PEPTIDASE_REGULATOR_{—} | METAB_OF_XENOBIOTICS_{—} |
| | RESPONSE | ACTIVITY | BY_P450 |
| #3 | ANTIMICROBIAL_{—} | FIBRONECTIN_BINDING | COMPLEMENT_AND_{—} |
| | HUMORAL_RESPONSE | | COAGULATION |
| #4 | MONOCYTE_CHEMOTAXIS | ENDOPEPTIDASE_{—} | ECM_RECEPTOR_{—} |
| | | REGULATOR | INTERACTION |
| #5 | LEUKOCYTE_MIGRATION | CHEMOKINE_RECEPTOR_{—} | NOD_LIKE_RECEPTOR_{—} |
| | | BINDING | SIGNALING |
| #6 | NEGATIVE_REG_OF_{—} | CHEMOKINE_RECEPTOR_{—} | HEMATOPOIETIC_CELL_{—} |
| | PEPTIDASE_ACTIVITY | BINDING | LINEAGE |
| #7 | NEUTROPHIL_{—} | GROWTH_FACTOR_{—} | DRUG_METAB_CYTOCHROME_{—} |
| | CHEMOTAXIS | ACTIVITY | P450 |
| #8 | ACUTE_INFLAMMATORY_{—} | CYTOKINE_RECEPTOR_{—} | TYROSINE_METAB |
| | RESPONSE | BINDING | |
| #9 | DEFENSE_RESPONSE_TO_{—} | ECM_STRUCTURAL_1 | RIBOSOME |
| | BACTERIUM | | |
| #10 | REG_OF_HUMORAL_{—} | ECM_STRUCTURAL_2 | PRIMARY_{—} |
| | IMMUNE_RESPONSE | | IMMUNODEFICIENCY |

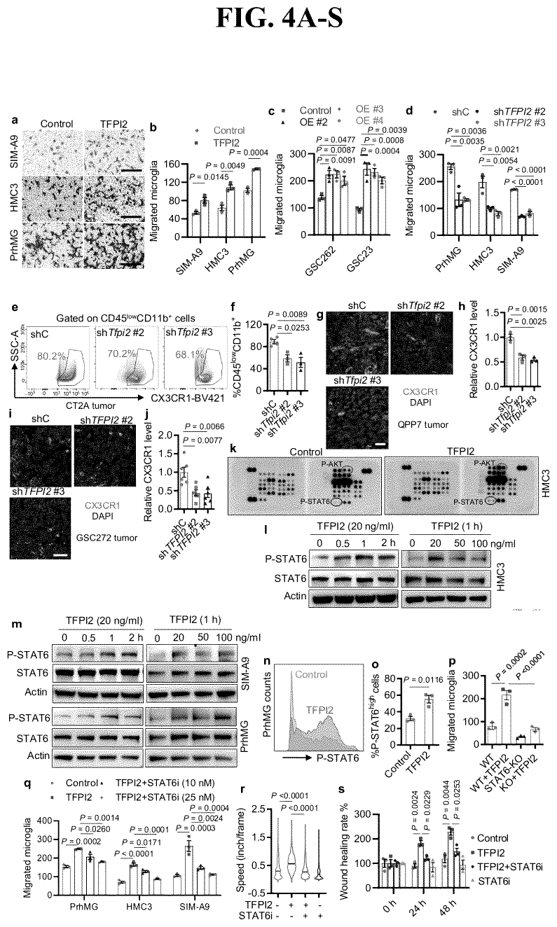

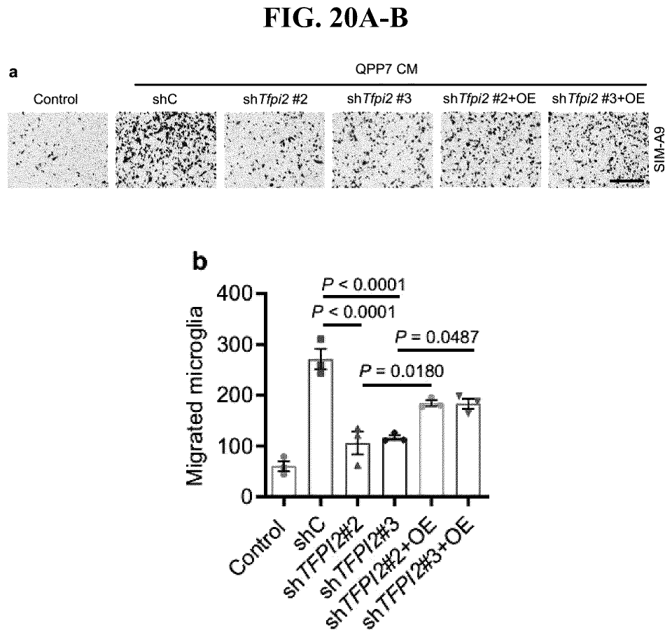

Since TFPI2 is a secreted protein, experiments were conducted during development of embodiments herein to determine whether GSC-derived TFPI2 promote microglia infiltration. Using transwell migration assays, we found that recombinant TFPI2-supplemented media dramatically increased the migration of distinct types of microglia (FIG. 4a,b), including mouse (SIM-A9) and human (HMC3) microglia cell lines, and primary human microglia (PrhMG, isolated from postmortem human brains (Ref. 38; incorporated by reference in its entirety)). To further investigate the role of TFPI2 in promoting microglia infiltration in the context of GBM, the effects of conditioned media (CM) from TFPI2-overexpressing GSC262 and GSC23 were examined, with results showing enhanced migration of PrhMG (FIG. 4c and FIG. 12c). CM from TFPI2-depleted GSC272 and QPP7 GSCs reduced the migration of human and mouse microglia (FIG. 4d and FIG. 12d). The impaired microglia migration was partially rescued when CM from TFPI2-depleted GSCs transfected with shRNA-resistant TFPI2 cDNA was used (FIG. 20a,b). To investigate TFPI2's effect on microglia infiltration in vivo, microglia abundance was determined in GBM tumors established from GSCs with and without shTFPI2. Flow cytometry results showed that TFPI2 depletion significantly reduced the population of CD45^{low}CD11b⁺ cells, but had little effect on CD45^{high}CD11b⁺ cells in CT2A tumors (FIG. 12e,f). The reduction effect was specific to CD45^{low}CD11b⁺CX3CR1⁺ microglia (FIG. 4e,f), but not CD45^{high}CD11b⁺CD68⁺ macrophages (FIG. 12g,h). Immunofluorescence for CX3CR1 confirmed that TFPI2 depletion reduced microglia abundance in QPP7 tumors (FIG. 4g,h) and GSC272 tumors (FIG. 4i,j). These findings demonstrate that TFPI2 is a novel chemokine triggering microglia infiltration into the GBM TME.

To investigate the molecular basis of TFPI2-induced microglia migration, a human phospho-kinase antibody array was used, the results from which showed STAT6 and AKT as being activated in HMC3 microglia in association with TFPI2 recombinant protein treatment (FIG. 4k). Bioinformatics analyses in TCGA GBM tumors demonstrated that STAT6, but not AKT1 and AKT2, positively correlated with microglia signature (FIG. 12i). Moreover, STAT6 showed a stronger positive correlation with microglia signature than AKT3 (FIG. 12i). Immunoblotting and flow cytometry results confirmed increased phosphorylation of STAT6 in human and mouse microglia upon the treatment with TFPI2 recombinant protein (FIG. 4l-o).

To further investigate the role of STAT6 in mediating TFPI2-induced microglia migration, microglia were isolated from wild-type (WT) and STAT6-KO mice and used in a transwell migration assay, the results of which showed that microglia isolated from STAT6-KO mice had impaired migration ability compared to microglia isolated from WT mice in response to TFPI2 treatment (FIG. 4p and FIG. 12j). Pharmacologic inhibition of STAT6 with AS1517499 suppressed TFPI2-induced transwell migration of human and mouse microglia (FIG. 4q and FIG. 12k). The impairment of TFPI2-induced HMC3 microglia migration from AS1517499 treatment was also confirmed by the investigations using incucyte live-cell and scratch assays (FIG. 4r,s, FIG. 21). These findings support a critical role for STAT6 signaling in mediating TFPI2-directed microglia infiltration into the GBM TME.

### The TFPI2-STAT6 Signaling Axis Triggers Microglia Immunosuppressive Polarization.

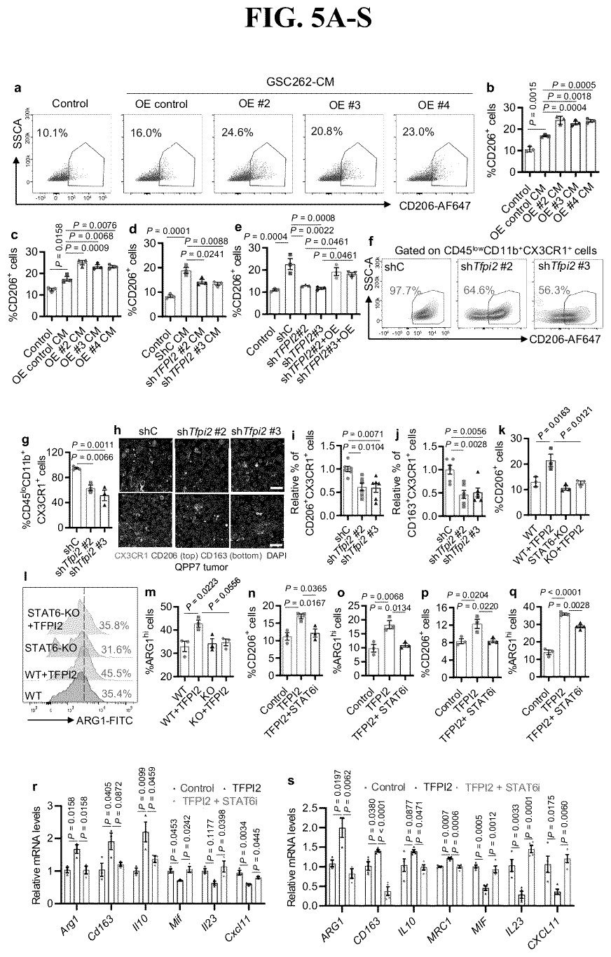

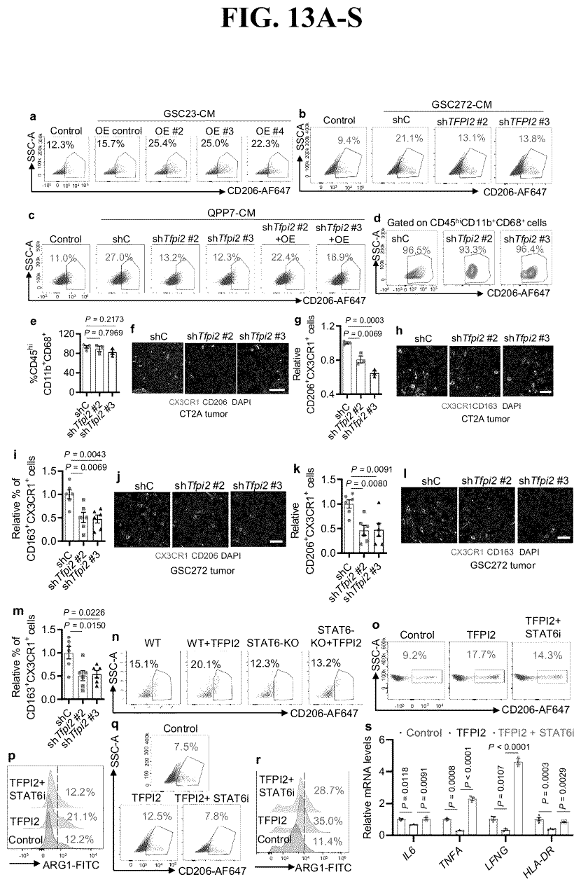

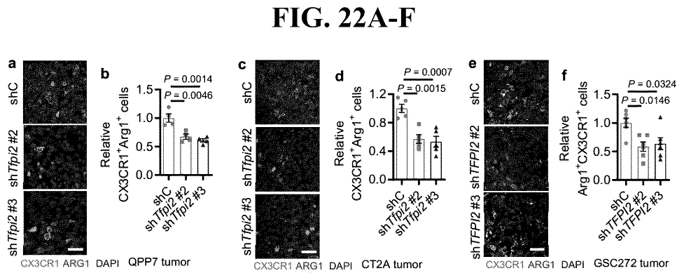

Microglia are a heterogeneous population of immune cells in the GBM TME and can be classified as immunostimulatory or immunosuppressive phenotype (Ref. 8; incorporated by reference in its entirety). Flow cytometry results showed that CM from GSC262 and GSC23 polarized PrhMG towards an immunosuppressive phenotype by increasing the percentage of CD206⁺ cells, and this effect was increased when using CM from GSC262 and GSC23 modified for increasing TFPI2 expression (FIG. 5a-c and FIG. 13a). In contrast, microglia immunosuppressive polarization was inhibited when treating with CM from TFPI2-depleted GSCs (FIG. 5d,e and FIG. 13b,c). The impaired microglia immunosuppressive polarization was rescued by reexpression of shRNA-resistant TFPI2 cDNA in shTFPI2 GSCs (FIG. 5e and FIG. 13c). In vivo, TFPI2 depletion in tumors established from CT2A cells presented reduced numbers of CD45^{low}CD11b⁺CX3CR1⁺CD206⁺ immunosuppressive microglia (FIG. 5f,g), while not affecting CD45^{high}CD11b⁺CD68⁺CD206⁺ immunosuppressive macrophages (FIG. 13d,e). Co-immunofluorescence for CX3CR1 and immunosuppressive markers (e.g., CD206, CD163, and ARG1) confirmed that immunosuppressive microglia were reduced upon TFPI2 depletion in tumors established from QPP7 GSCs (FIG. 5h-j and FIG. 22a,b), CT2A cells (FIG. 13f-i and FIG. 22c,d), and GSC272 (FIG. 13j-m and FIG. 22e,f). These findings demonstrate that GSC-derived TFPI2 is essential for microglia immunosuppressive polarization in GBM.

Experiments were conducted during development of embodiments herein to investigate whether TFPI2-induced microglia immunosuppressive polarization is regulated by STAT6 signaling using flow cytometry analysis for CD206 and ARG1 expression, with results showing that immunosuppressive microglia were increased by TFPI2 recombinant protein treatment. This increase was negated by genetic depletion of STAT6 in primary mouse microglia (FIG. 5k-m and FIG. 13n) and by pharmacologic inhibition of STAT6 using AS1517499 in both mouse and human microglia (FIG. 5n-q and FIG. 13o-r). This result is consistent with data obtained from RT-qPCR for immunosuppressive marker expression in mouse and human microglia (FIG. 5r,s). A role of the TFPI2-STAT6 signaling pathway in regulating microglia immunostimulatory polarization was examined using RT-qPCR, with results showing that TFPI2 recombinant protein treatment suppressed the expression of immunostimulatory microglia markers (e.g., MIF, IL23, CXCL11, IL6, TNFA, LFNG, and HLA-DR) in microglia, which was countered by treatment with STAT6 inhibitor AS1517499 (FIG. 5r,s and FIG. 13s). These findings indicate that STAT6 signaling is involved in TFPI2-induced microglia immunosuppressive polarization in GBM.

CD51 is a Functional TFPI2 Receptor that Mediates TFPI2-Induced Microglia Migration and Immunosuppressive Polarization.

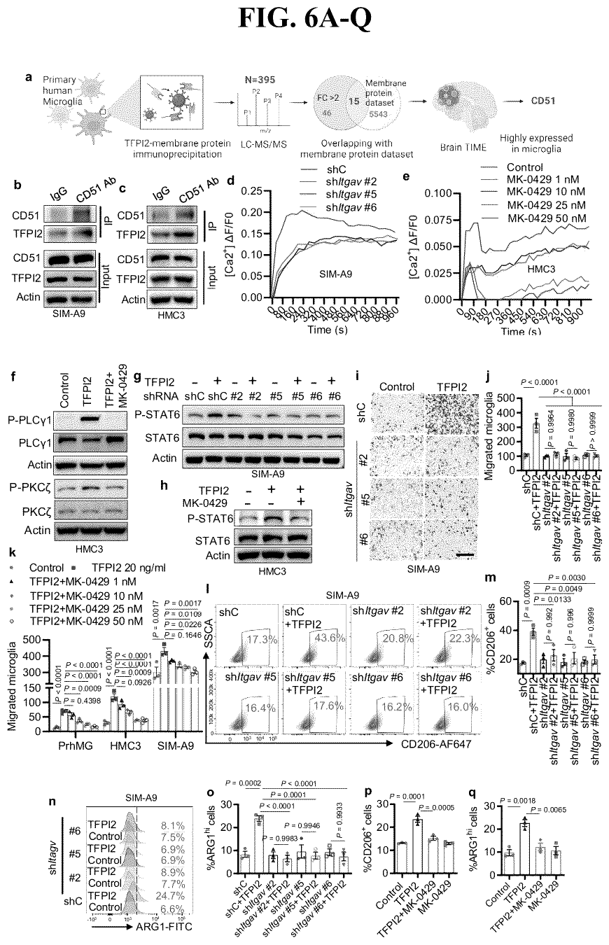

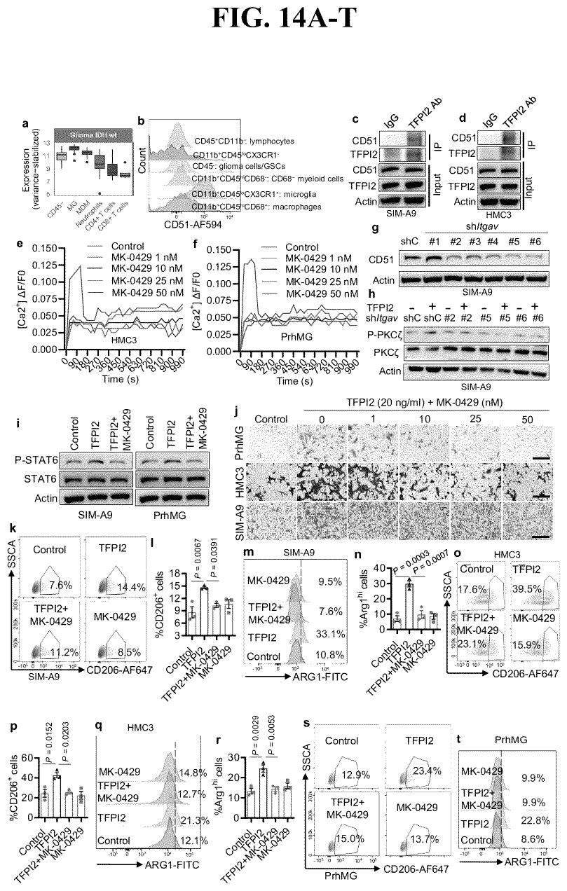

To investigate whether TFPI2 exerts migratory and polarization effects through interaction with microglia membrane proteins, PrhMG were incubated with TFPI2 recombinant protein (FIG. 6a). Membranous PrhMG proteins were subsequently extracted and TFPI2 was immunoprecipitated (FIG. 6a). The major protein band revealed by denaturing gel electrophoresis of immunoprecipitates was analyzed by liquid chromatography-mass spectrometry (LC-MS), with results showing 395 co-precipitated proteins. Sixty-one of them exhibited a ≥2-fold change in TFPI2 IP group versus control IgG group. Searching for these proteins in a membrane protein database (ref. 40; incorporated by reference in its entirety) revealed 15 membrane protein candidates as potentially interacting with TFPI2 (FIG. 6a). With further filtering using the TIME dataset of IDH-WT GBM patient tumors (Ref. 29; incorporated by reference in its entirety) as well as data from CT2A tumor model, CD51 was identified as the only potential receptor exhibiting higher expression in microglia compared to CD45⁻ GBM cells, macrophages, lymphocytes, and other immune cells (FIG. 14a,b). The interaction between CD51 and TFPI2 was confirmed by immunoblotting of mouse and human microglia immunoprecipitates with an anti-CD51 antibody (FIG. 6b,c) or anti-TFPI2 antibody (FIG. 14c,d). To determine whether CD51 affects TFPI2 signaling in microglia, a calcium mobilization assay was utilized, which has been widely used to measure the activation of receptors, including chemokine receptors (Refs. 41-42; incorporated by reference in their entireties), with results showing that genetic and pharmacologic inhibition of CD51 decreased TFPI2-triggered calcium mobilization in both mouse and human microglia (FIG. 6d,e and FIG. 14e,f). TFPI2-induced phosphorylation of PLCγ1 and PKCQ, known mediators in the calcium signaling pathway, was also inhibited by genetic depletion and pharmacologic inhibition of CD51 in microglia (FIG. 6f and FIG. 14g,h).

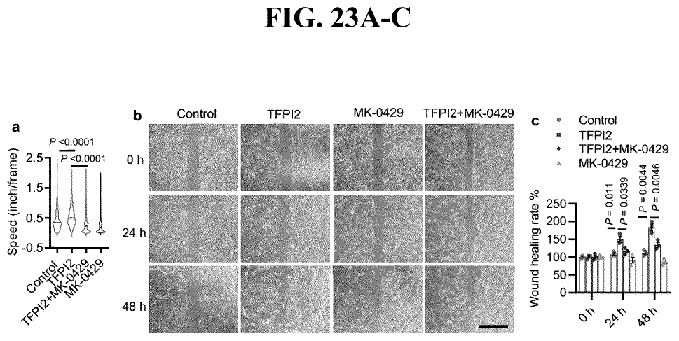

Experiments were conducted during development of embodiments herein to examine whether CD51 inhibition influences TFPI2 effects on microglia. TFPI2-induced phosphorylation of STAT6 was impaired by genetic and pharmacologic inhibition of CD51 in mouse and human microglia (FIG. 6g,h and FIG. 14i), and this effect was accompanied by reduced microglia migration as indicated by the results from transwell, incucyte live-cell, and scratch assays (FIG. 6i-k, FIG. 14j, FIG. 23a-c and). CD51 inhibition also reduced TFPI2-induced microglia immunosuppressive polarization as indicated by reduced expression of immunosuppressive markers CD206 and ARG1 in mouse and human microglia (FIG. 6l-q and FIG. 14k-t). These findings indicate that CD51 is a functional receptor of TFPI2 that is essential for TFPI2-induced microglia migration and immunosuppressive polarization.

Inhibition of the TFPI2-CD51-STAT6 Signaling Axis Activates Antitumor Immune Response and Synergizes with Anti-PD1 Therapy.

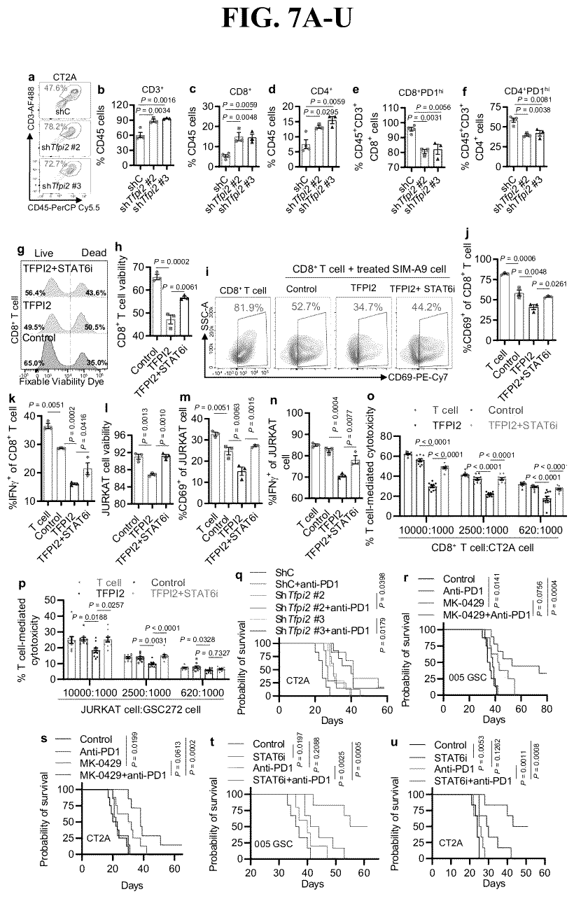

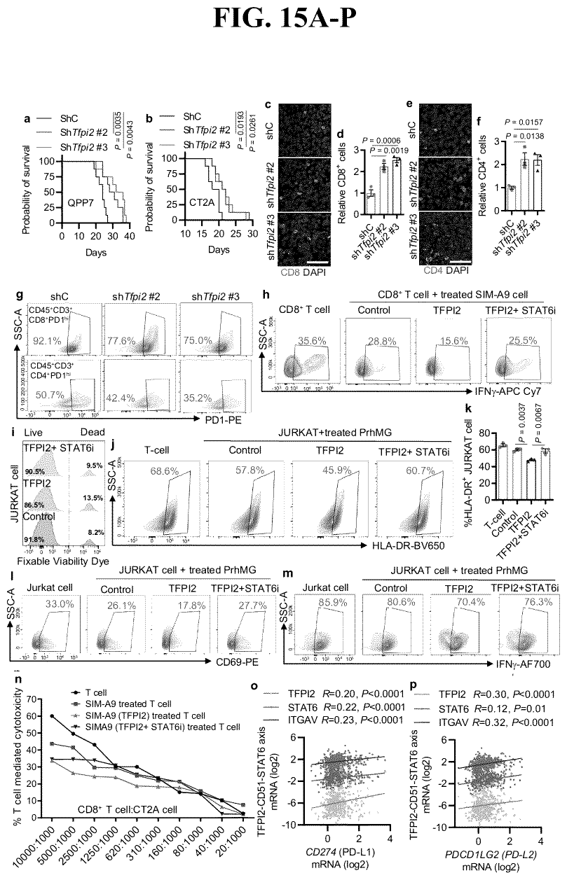

Experiments were conducted during development of embodiments herein to determine whether inhibiting microglia immunosuppressive effects via blockade of the TFPI2-CD51-STAT6 signaling pathway would influence T cell-mediated anti-tumor immune responses in GBM. shC and shTfpi2 QPP7 GSCs and CT2A cells were implanted into the brains of immunocompromised nude mice. The anti-tumor effects associated with TFPI2 depletion were reduced in nude mice when compared against results from immunocompetent C57BL/6 mice (FIG. 15a,b and FIG. 2n,o). Flow cytometry was performed on spleens from CT2A tumor-bearing C57BL/6 mice and it was found that TFPI2 depletion significantly increased CD3⁺ (CD45⁺CD3⁺), CD8⁺ (CD45⁺CD3⁺CD8⁺), and CD4⁺ (CD45⁺CD3⁺CD4⁺) T cell populations (FIG. 7a-d). Increases of CD8⁺ and CD4⁺ T cells were confirmed by immunofluorescence staining of TFPI2-depleted CT2A and QPP7 tumors (FIG. 15c-f). Furthermore, the exhausted T cells (e.g., CD45⁺CD3⁺CD8⁺PD1^{high} and CD45⁺CD3⁺CD4⁺PD1^{high} cells) were downregulated in the spleens of CT2A tumor-bearing mice upon TFPI2 depletion (FIG. 7e,f and FIG. 15g). These findings indicate that TFPI2 depletion can increase T cell infiltration and activation in tumors of immunocompetent GBM mouse models. To confirm the involvement of microglia with these results, a mouse microglia and CD8⁺ T cell co-culture approach was utilized to demonstrate that TFPI2-polarized microglia decreased CD8⁺ T cell viability. This effect was negated by treatments with STAT6 inhibitor AS1517499 (FIG. 7g,h). It was also found that the percentage of CD8⁺CD69⁺ and CD8⁺IFNγ⁺ T cells was reduced when they were co-cultured with TFPI2-treated microglia. These reductions were prevented by AS1517499 treatments (FIG. 7i-k and FIG. 15h). Results from human microglia-T cell co-cultures also showed that TFPI2-polarized microglia suppressed JURKAT (human CD4⁺CD8⁻) T cell viability and activation, and these effects were prevented by AS1517499 treatments (FIG. 7l-n and FIG. 15i-m). Functionally, TFPI2-polarized microglia suppressed T cell-mediated tumor cell cytotoxicity compared to control microglia, and this immunosuppressive function was rescued by AS1517499 treatments (FIG. 7o,p and FIG. 15n). These findings indicate that blockade of microglia immunosuppressive polarization via inhibition of the TFPI2-STAT6 signaling pathway can increase T cell infiltration, activation, and cytotoxicity.

Since the CD51-STAT6 signaling axis plays a prominent role in promoting TFPI2-induced microglia migration, immunosuppressive polarization, and anti-tumor immune response, experiments were conducted during development of embodiments herein to explore the impact of this signaling on tumor growth and ICI therapy efficacy in immunocompetent GBM mouse models. A second immunocompetent GBM mouse model, 005 GSC, that expresses relatively high levels of TFPI2 (FIG. 1e) and has been reported as non-responsive to immunotherapy was utilized. Genetic and pharmacological inhibition of the TFPI2-CD51-STAT6 signaling axis extended the survival of C57BL/6 mice implanted with 005 GSCs and CT2A cells (FIG. 7q-u). TCGA GBM data analysis showed that the expression of PD-L1 and PD-L2 correlated positively with TFPI2, ITGAV, and STAT6 expression (FIG. 15o,p). Experiments were conducted during development of embodiments herein to investigate combined TFPI2-CD51-STAT6 inhibition plus anti-PD1 therapy in GBM-bearing mice. The results showed that depletion of TFPI2 in GSCs (FIG. 7q), treatment with CD51 inhibitor MK-0429 (FIG. 7r,s), or treatment with STAT6 inhibitor AS1517499 (FIG. 7t,u) synergized with anti-PD1 therapy to extend the survival of animal subjects bearing 005 GSC and CT2A tumors. These findings indicate that inhibition of the TFPI2-CD51-STAT6 signaling axis activates antitumor immune response that can be exploited through combination with anti-PD1 treatment in GBM.

TFPI2 is a Prognostic Biomarker that Correlates with GSC Stemness and Microglia Abundance in GBM.

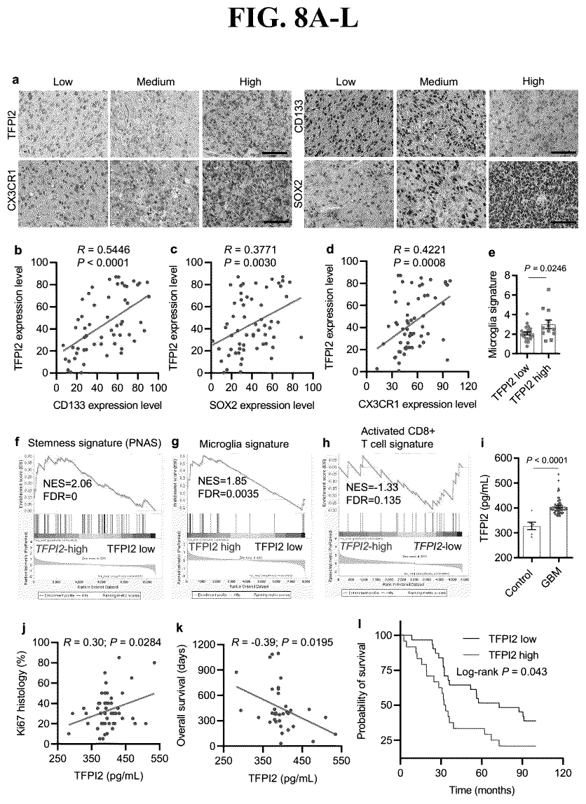

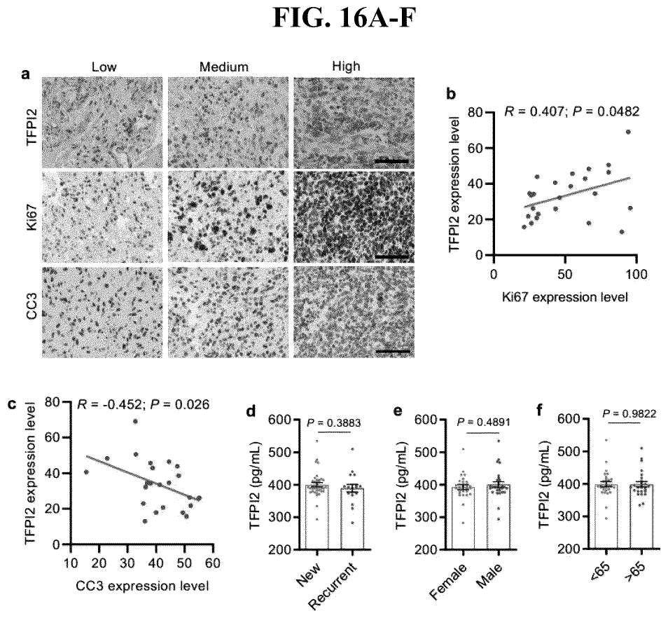

Immunohistochemistry staining for TFPI2, SOX2, CD133, CX3CR1, Ki67, and cleaved caspase 3 was performed in GBM patient tumors (FIG. 8a and FIG. 16a). The analysis revealed that the expression of TFPI2 correlated positively with CD133 (FIG. 8b), SOX2 (FIG. 8c), Ki67 (FIG. 16b), and CX3CR1 (FIG. 8d), and correlated negatively with cleaved caspase 3 (FIG. 16c), supporting that high TFPI2 expression is associated with increased GSC sternness, proliferation, survival, and microglia abundance in GBM patient tumors. Analysis of scRNA-seq data from GBM patient tumors confirmed that elevated TFPI2 expression in GBM cells correlated with increased microglia signature (FIG. 8e). Analysis of TCGA GBM patient data showed that stemness and microglia signatures were enriched in TFPI2-high patient tumors (FIG. 8f,g), whereas activated CD8⁺ T cell signature was enriched in TFPI2-low patient tumors (FIG. 8h).

Since TFPI2 encodes a secreted protein, its protein level was analyzed in the plasma of 6 healthy controls and 55 GBM patients, with results showing that plasma levels of TFPI2 were significantly higher in GBM patients than healthy controls (FIG. 8i). Moreover, correlation analyses revealed that plasma TFPI2 is correlated positively with intratumoral Ki67 expression (FIG. 8j) and negatively with patient overall survival (FIGS. 8k,l). The analyses also showed that TFPI2 levels were not correlated with status of recurrence, gender, and age of GBM patients (FIG. 16d-f). These human GBM findings confirm the role of TFPI2 in regulation of GSC stemness, microglia infiltration, and T cell activation, and indicate that TFPI2 is a prognostic biomarker for GBM patients.

### REFERENCE

The following references, some of which are cited above, are herein incorporated by reference in their entireties.
1. Lim, M., Xia, Y., Bettegowda, C. & Weller, M. Current state of immunotherapy for glioblastoma. Nature Reviews Clinical Oncology 15, 422-442 (2018).
2. Prager, B. C., Bhargava, S., Mahadev, V., Hubert, C. G. & Rich, J. N. Glioblastoma Stem Cells: Driving Resilience through Chaos. Trends in Cancer 6, 223-235 (2020).
3. Couturier, C. P. et al. Single-cell RNA-seq reveals that glioblastoma recapitulates a normal neurodevelopmental hierarchy. Nature Communications 11, 3406 (2020).
4. Guilhamon, P. et al. Single-cell chromatin accessibility profiling of glioblastoma identifies an invasive cancer stem cell population associated with lower survival. eLife 10, e64090 (2021).
5. Tao, W. et al. Dual Role of WISP1 in maintaining glioma stem cells and tumor-supportive macrophages in glioblastoma. Nature Communications 11, 3015 (2020).
6. Fang, X. et al. Inhibiting DNA-PK induces glioma stem cell differentiation and sensitizes glioblastoma to radiation in mice. Science Translational Medicine 13, eabc7275 (2021).
7. Chen, P., Hsu, W. H., Han, J., Xia, Y. & DePinho, R. A. Cancer Stemness Meets Immunity: From Mechanism to Therapy. Cell Rep 34, 108597 (2021).
8. Xuan, W., Lesniak, M. S., James, C. D., Heimberger, A. B. & Chen, P. Context-Dependent Glioblastoma-Macrophage/Microglia Symbiosis and Associated Mechanisms. Trends in Immunology 42, 280-292 (2021).
9. Zhou, W. et al. Periostin secreted by glioblastoma stem cells recruits M2 tumour-associated macrophages and promotes malignant growth. Nat Cell Biol 17, 170-182 (2015).
10. Shi, Y. et al. Tumour-associated macrophages secrete pleiotrophin to promote PTPRZ1 signalling in glioblastoma stem cells for tumour growth. Nat Commun 8, 15080 (2017).
11. Xuan, W. et al. Circadian Regulator CLOCK Drives Immunosuppression in Glioblastoma. Cancer Immunology Research 10, 770-784 (2022).
12. Chen, P. et al. Circadian Regulator CLOCK Recruits Immune-Suppressive Microglia into the GBM Tumor Microenvironment. Cancer Discovery 10, 371-381 (2020).
13. Sprecher, C. A., Kisiel, W., Mathewes, S. & Foster, D. C. Molecular cloning, expression, and partial characterization of a second human tissue-factor-pathway inhibitor. Proceedings of the National Academy of Sciences 91, 3353-3357 (1994).
14. Herman, M. P. et al. Tissue factor pathway inhibitor-2 is a novel inhibitor of matrix metalloproteinases with implications for atherosclerosis. The Journal of Clinical Investigation 107, 1117-1126 (2001).
15. Winkler, J., Abisoye-Ogunniyan, A., Metcalf, K. J. & Werb, Z. Concepts of extracellular matrix remodelling in tumour progression and metastasis. Nature Communications 11, 5120 (2020).
16. Lavergne, M. et al. Beneficial role of overexpression of TFPI-2 on tumour progression in human small cell lung cancer. FEBS Open Bio 3, 291-301 (2013).
17. Wang, G. et al. TFPI-2 suppresses breast cancer cell proliferation and invasion through regulation of ERK signaling and interaction with actinin-4 and myosin-9. Scientific Reports 8, 14402 (2018).
18. Neaud, V. et al. Paradoxical Pro-invasive Effect of the Serine Proteinase Inhibitor Tissue Factor Pathway Inhibitor-2 on Human Hepatocellular Carcinoma Cells*. Journal of Biological Chemistry 275, 35565-35569 (2000).
19. Ruf, W. et al. Differential Role of Tissue Factor Pathway Inhibitors 1 and 2 in Melanoma Vasculogenic Mimicryl. Cancer Research 63, 5381-5389 (2003).
20. Mo, J. et al. TFPI2 Promotes Perivascular Migration in an Angiotropism Model of Melanoma. Front Oncol 11, 662434 (2021).
21. Ota, Y. et al. Tissue factor pathway inhibitor-2 is specifically expressed in ovarian clear cell carcinoma tissues in the nucleus, cytoplasm and extracellular matrix. Oncol Rep 45, 1023-1032 (2021).
22. George, J., Gondi, C. S., Dinh, D. H., Gujrati, M. & Rao, J. S. Restoration of Tissue Factor Pathway Inhibitor-2 in a Human Glioblastoma Cell Line Triggers Caspase-Mediated Pathway and Apoptosis. Clinical Cancer Research 13, 3507-3517 (2007).
23. Gessler, F., Voss, V., Seifert, V., Gerlach, R. & Kogel, D. Knockdown of TFPI-2 promotes migration and invasion of glioma cells. Neurosci Lett 497, 49-54 (2011).
24. Rao, C. N. et al. Expression of tissue factor pathway inhibitor 2 inversely correlates during the progression of human gliomas. Clin Cancer Res 7, 570-576 (2001).
25. Miranda, A. et al. Cancer stemness, intratumoral heterogeneity, and immune response across cancers. Proc Natl Acad Sci USA 116, 9020-9029 (2019).
26. Yoshihara, K. et al. Inferring tumour purity and stromal and immune cell admixture from expression data. Nat Commun 4, 2612 (2013).
27. Chai, R. et al. A novel gene signature based on five glioblastoma stem-like cell relevant genes predicts the survival of primary glioblastoma. J Cancer Res Clin Oncol 144, 439-447 (2018).
28. Uhlen, M. et al. The human secretome. Sci Signal 12 (2019).
29. Klemm, F. et al. Interrogation of the Microenvironmental Landscape in Brain Tumors Reveals Disease-Specific Alterations of Immune Cells. Cell 181, 1643-1660 e1617 (2020).
30. Neftel, C. et al. An Integrative Model of Cellular States, Plasticity, and Genetics for Glioblastoma. Cell 178, 835-849 e821 (2019).
31. Shingu, T. et al. Qki deficiency maintains sternness of glioma stem cells in suboptimal environment by downregulating endolysosomal degradation. Nature Genetics 49, 75-86 (2017).
32. Saha, D., Martuza, R. L. & Rabkin, S. D. Macrophage Polarization Contributes to Glioblastoma Eradication by Combination Immunovirotherapy and Immune Checkpoint Blockade. Cancer Cell 32, 253-267 e255 (2017).
33. Yoon, C. H. et al. c-Jun N-terminal kinase has a pivotal role in the maintenance of self-renewal and tumorigenicity in glioma stem-like cells. Oncogene 31, 4655-4666 (2012).
34. Gu, C. Y. et al. Tumor-Specific Activation of the C-JUN/MELK Pathway Regulates Glioma Stem Cell Growth in a p53-Dependent Manner. Stem Cells 31, 870-881 (2013).
35. Shi, Y. et al. Ibrutinib inactivates BMX-STAT3 in glioma stem cells to impair malignant growth and radioresistance. Sci Transl Med 10 (2018).
36. Stechishin, O. D. et al. On-target JAK2/STAT3 inhibition slows disease progression in orthotopic xenografts of human glioblastoma brain tumor stem cells. Neuro Oncol 15, 198-207 (2013).
37. Deng, Y. et al. Identifying mutual exclusivity across cancer genomes: computational approaches to discover genetic interaction and reveal tumor vulnerability. Brief Bioinform 20, 254-266 (2019).
38. Guo, L. et al. Postmortem Adult Human Microglia Proliferate in Culture to High Passage and Maintain Their Response to Amyloid-beta. J Alzheimers Dis 54, 1157-1167 (2016).
39. Nagashima, S. et al. Synthesis and evaluation of 2-{[2-(4-hydroxyphenyl)-ethyl]amino}pyrimidine-5-carboxamide derivatives as novel STAT6 inhibitors. Bioorgan Med Chem 15, 1044-1055 (2007).
40. Uhlen, M. et al. Tissue-based map of the human proteome. Science 347 (2015).
41. Chen, P. et al. Gpr132 sensing of lactate mediates tumor-macrophage interplay to promote breast cancer metastasis. Proc Natl Acad Sci USA 114, 580-585 (2017).
42. Chen, J. et al. CCL18 from tumor-associated macrophages promotes breast cancer metastasis via PITPNM3. Cancer Cell 19, 541-555 (2011).
43. Chen, C. H. et al. Novel Murine Glioblastoma Models That Reflect the Immunotherapy Resistance Profile of Human Disease. Neuro Oncol (2023).
44. Bloch, O. et al. Gliomas promote immunosuppression through induction of B7-H1 expression in tumor-associated macrophages. Clin Cancer Res 19, 3165-3175 (2013).
45. Gimple, R. C., Yang, K. L., Halbert, M. E., Agnihotri, S. & Rich, J. N. Brain cancer stem cells: resilience through adaptive plasticity and hierarchical heterogeneity. Nature Reviews Cancer (2022).
46. Wang, X. et al. Sequential fate-switches in stem-like cells drive the tumorigenic trajectory from human neural stem cells to malignant glioma. Cell Res 31, 684-702 (2021).
47. Bachoo, R. M. et al. Epidermal growth factor receptor and Ink4a/Arf: convergent mechanisms governing terminal differentiation and transformation along the neural stem cell to astrocyte axis. Cancer Cell 1, 269-277 (2002).
48. Dong, Z. et al. Targeting Glioblastoma Stem Cells through Disruption of the Circadian Clock. Cancer Discov 9, 1556-1573 (2019).
49. Sato, N. et al. Epigenetic inactivation of TFPI-2 as a common mechanism associated with growth and invasion of pancreatic ductal adenocarcinoma. Oncogene 24, 850-858 (2005).
50. Rollin, J. et al. Expression and methylation status of tissue factor pathway inhibitor-2 gene in non-small-cell lung cancer. British Journal of Cancer 92, 775-783 (2005).
51. Fullar, A. et al. Two ways of epigenetic silencing of TFPI2 in cervical cancer. PLOS ONE 15, e0234873 (2020).
52. Dirkse, A. et al. Stem cell-associated heterogeneity in Glioblastoma results from intrinsic tumor plasticity shaped by the microenvironment. Nature Communications 10, 1787 (2019).
53. Schaettler, M. O. et al. Characterization of the Genomic and Immunologic Diversity of Malignant Brain Tumors through Multisector Analysis. Cancer Discovery 12, 154-171 (2021).
54. Konduri, S. D. et al. A novel function of tissue factor pathway inhibitor-2 (TFPI-2) in human glioma invasion. Oncogene 20, 6938-6945 (2001).
55. Jacob, F. et al. A Patient-Derived Glioblastoma Organoid Model and Biobank Recapitulates Inter- and Intra-tumoral Heterogeneity. Cell 180, 188-204.e122 (2020).
56. LeBlanc, V. G. et al. Single-cell landscapes of primary glioblastomas and matched explants and cell lines show variable retention of inter- and intratumor heterogeneity. Cancer Cell 40, 379-392.e379 (2022).
57. Bayik, D. & Lathia, J. D. Cancer stem cell-immune cell crosstalk in tumour progression. Nature Reviews Cancer 21, 526-536 (2021).
58. Pang, L., Khan, F., Dunterman, M. & Chen, P. Pharmacological targeting of the tumor-immune symbiosis in glioblastoma. Trends in Pharmacological Sciences 43, 686-700 (2022).
59. Brennan, C. W. et al. The Somatic Genomic Landscape of Glioblastoma. Cell 155, 462-477 (2013).
60. Parsa, A. T. et al. Loss of tumor suppressor PTEN function increases B7-H1 expression and immunoresistance in glioma. Nature Medicine 13, 84-88 (2007).
61. Chen, P. et al. Symbiotic Macrophage-Glioma Cell Interactions Reveal Synthetic Lethality in PTEN-Null Glioma. Cancer Cell 35, 868-884.e866 (2019).
62. Liu, H., Wang, G., Hao, D., Wang, C. & Zhang, M. Antimicrobial and Immunoregulatory Activities of TS40, a Derived Peptide of a TFPI-2 Homologue from Black Rockfish (Sebastes schlegelii). Mar Drugs 20 (2022).
63. Cai, W. et al. STAT6/Arg1 promotes microglia/macrophage efferocytosis and inflammation resolution in stroke mice. Jci Insight 4 (2019).
64. Wang, N., Liang, H. & Zen, K. Molecular mechanisms that influence the macrophage m1-m2 polarization balance. Front Immunol 5, 614 (2014).
65. Chen, P. et al. Symbiotic Macrophage-Glioma Cell Interactions Reveal Synthetic Lethality in PTEN-Null Glioma. Cancer Cell 35, 868-884 e866 (2019).
66. Pang, L., Khan, F., Heimberger, A. B. & Chen, P. Mechanism and therapeutic potential of tumor-immune symbiosis in glioblastoma. Trends in Cancer 8, 839-854 (2022).
67. Zhang, B. et al. Macrophage-expressed CD51 promotes cancer stem cell properties via the TGF-01/smad2/3 axis in pancreatic cancer. Cancer Letters 459, 204-215 (2019).
68. Chen, P. & Dey, P. Co-dependencies in the tumor immune microenvironment. Oncogene 41, 3821-3829 (2022).
69. Chamberlain, M. C. & Kim, B. T. Nivolumab for patients with recurrent glioblastoma progressing on bevacizumab: a retrospective case series. J Neurooncol 133, 561-569 (2017).
70. Reiss, S., Yerram, P., Modelevsky, L. & Grommes, C. Retrospective review of safety and efficacy of checkpoint inhibition in refractory high-grade gliomas. J Clin Oncol 35 (2017).
71. Reardona, D. A. et al. Randomized Phase 3 Study Evaluating the Efficacy and Safety of Nivolumab Vs Bevacizumab in Patients with Recurrent Glioblastoma: Checkmate 143. Neuro-oncology 19, 21-21 (2017).
72. Zhao, J. et al. Immune and genomic correlates of response to anti-PD-1 immunotherapy in glioblastoma. Nat Med 25, 462-469 (2019).
73. Arrieta, V. A. et al. ERK1/2 phosphorylation predicts survival following anti-PD-1 immunotherapy in recurrent glioblastoma. Nat Cancer 2, 1372-1386 (2021).
74. Chen, P. et al. Collagen VI regulates peripheral nerve regeneration by modulating macrophage recruitment and polarization. Acta Neuropathol 129, 97-113 (2015).
75. Ershov, D. et al. TrackMate 7: integrating state-of-the-art segmentation algorithms into tracking pipelines. Nat Methods 19, 829-832 (2022).
76. Tinevez, J. Y. et al. TrackMate: An open and extensible platform for single-particle tracking. Methods 115, 80-90 (2017).
77. Varghese, F., Bukhari, A. B., Malhotra, R. & De, A. IHC Profiler: An Open Source Plugin for the Quantitative Evaluation and Automated Scoring of Immunohistochemistry Images of Human Tissue Samples. PLOS ONE 9, e96801 (2014).
78. Barth, N. D. et al. A fluorogenic cyclic peptide for imaging and quantification of drug-induced apoptosis. Nature Communications 11, 4027 (2020).
79. Ong Derrick Sek, T. et al. PAF promotes stemness and radioresistance of glioma stem cells. Proceedings of the National Academy of Sciences 114, E9086-E9095 (2017).
80. Galaxy, C. The Galaxy platform for accessible, reproducible and collaborative biomedical analyses: 2022 update. Nucleic Acids Res 50, W345-W351 (2022).
81. Couturier, C. P. et al. Single-cell RNA-seq reveals that glioblastoma recapitulates a normal neurodevelopmental hierarchy. Nat Commun 11, 3406 (2020).

1. A method of treating cancer in a subject, wherein the subject has an inhibited TFPI2-CD51-STAT6 signaling axis, the method comprising administering to the subject an immune checkpoint inhibitor (ICI).

2. A method of treating cancer in a subject comprising administering a TFPI2 inhibitor to the subject.

3. The method of claim 2, further comprising co-administering to the subject
an immune checkpoint inhibitor (ICI).

4. The method of claim 2, wherein the subject suffers from glioblastoma.

5. The method of claim 3, wherein the ICI targets an immune checkpoint molecule/protein selected from the group consisting of CTLA4, PD-1, PD-L1, PD-L2, A2AR, B7-H3, B7-H4, BTLA, KIR, LAG3, TIM-3, VISTA, or a ligand/receptor thereof.

6. The method of claim 3, wherein the ICI is selected from nivolumab, pembrolizumab, atezolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

7. The method of claim 1, wherein the subject has been previously administered a TFPI2-CD51-STAT6 signaling axis inhibitor.

8. The method of claim 3, wherein the TFPI2-CD51-STAT6 signaling axis inhibitor is a STAT6 inhibitor.

9. The method of claim 8, wherein the STAT6 inhibitor is an inhibitor of STAT6 expression.

10. The method of claim 8, wherein the STAT6 inhibitor is an inhibitor of STAT6 activity.

11. The method of claim 3, wherein the TFPI2-CD51-STAT6 signaling axis inhibitor is a CD51 inhibitor.

12. The method of claim 11, wherein the CD51 inhibitor is an inhibitor of CD51 expression.

13. The method of claim 11, wherein the CD51 inhibitor is an inhibitor of CD51 activity.

14. The method of claim 3, wherein the TFPI2-CD51-STAT6 signaling axis inhibitor is a TFPI2 inhibitor.

15. The method of claim 2, wherein the TFPI2 inhibitor is an inhibitor of TFPI2 expression.

16. The method of claim 2, wherein the TFPI2 inhibitor is an inhibitor of TFPI2 activity.

17. The method of claim 9, 12, or 15, wherein the inhibitor of expression is an shRNA, a miRNA, a morpholino, a ribozyme, an antisense nucleic acid molecule, or a CRISPR-based construct.

18. The method of claim 16, wherein the inhibitor of activity is a small molecule, peptide, antibody, or antibody fragment.

19. The method of one of claim 2 wherein administration is by an oral or intravenous route.
